SEQUENCE LISTING (13 sequences)

SEQ ID NO: 1 - DNA, 20 bp, Artificial Sequence
  Synthesized Construct
ttgagtgcattctagttgtg

SEQ ID NO: 2 - DNA, 21 bp, Artificial Sequence
  Synthesized Construct
gtttcagtgcaatttatgcaa

SEQ ID NO: 3 - DNA, 20 bp, Artificial Sequence
  Synthesized Construct
ttatacatactatttggttg

SEQ ID NO: 4 - DNA, 20 bp, Artificial Sequence
  Synthesized Construct
gcctccaaaaagccaaacta

SEQ ID NO: 5 - DNA, 20 bp, Artificial Sequence
  Synthesized Construct
cacagtgatggggctgtatg

SEQ ID NO: 6 - DNA, 19 bp, Artificial Sequence
  Synthesized Construct
atgggtgaaactctgggag

SEQ ID NO: 7 - DNA, 19 bp, Artificial Sequence
  Synthesized Construct
ccttgcatccttcacaagc

SEQ ID NO: 8 - DNA, 20 bp, Artificial Sequence
  Synthesized Construct
gtacagttcacgcccctcac

SEQ ID NO: 9 - DNA, 20 bp, Artificial Sequence
  Synthesized Construct
tttgggagaggtgggaataa

SEQ ID NO: 10 - DNA, 21 bp, Artificial Sequence
  Synthesized Construct
cagggaattattgccatctga

SEQ ID NO: 11 - DNA, 20 bp, Artificial Sequence
  Synthesized Construct
ggcaaggaattgtggtcagt

SEQ ID NO: 12 - DNA, 20 bp, Artificial Sequence
  Synthesized Construct
cttgttgaataaatcacctt

SEQ ID NO: 13 - DNA, 20 bp, Artificial Sequence
  Synthesized Construct
cagtggcttttaaaatagaa

(19) United States
(12) Patent Application Publication
(10) Pub. No.: US 2010/0143330 A1
(43) Pub. Date: Jun. 10, 2010

(54) METHODS OF TREATING DISORDERS ASSOCIATED WITH FAT STORAGE

(75) Inventors: Isidro Sanchez-Garcia, Salamanca (ES); Pedro A. Perez-Mancera, Salamanca (ES)

Correspondence Address: MORRISON & FOERSTER LLP, 425 MARKET STREET, SAN FRANCISCO, CA, 94105-2482, US

(73) Assignees: UnversidadDe Salamanca (O.T.R.I.), Salamanca (ES); Consejo Superior De Investigaciones Cientificas, Salamanca (ES)

(21) Appl. No.: 12/445,928

(22) PCT Filed: Sep. 24, 2007
(86) PCT No.: PCT/IB07/03736

§ 371 Date: Dec. 7, 2009

(30) Foreign Application Priority Data
Oct. 16, 2006 (EP) 06076886.8

(51) Int. Cl. A61K 39/395; A61K 38/43; C12Q 1/68; C12N 9/00; A61K 38/16; A61K 31/7088; C07K 14/435; C07K 16/00; C07H 21/04; G01N 33/00; A61P 3/04

(52) U.S. Cl. 424/130.1; 424/94.1; 435/6; 435/183; 514/12; 514/44 A; 530/350; 530/387.1; 536/24.5; 800/3

(57) ABSTRACT

The invention relates, in general, to markers of obesity and lipodystrophy. In particular, the expression level of the SLUG gene or its expression products can be used as such a marker. Furthermore, the invention additionally relates to the use of SLUG as a therapeutic and diagnostic target for these pathologies. The invention further relates to a method of treating a disorder associated with increased or decreased fat storage in a mammal comprising modulating the activity or level of the SLUG protein or the SLUG gene in the mammal.

### FIELD OF INVENTION

The invention relates, in general, to markers of obesity and lipodystrophy. In particular, the expression level of the SLUG gene or its expression products can be used as such a marker. Furthermore, the invention additionally relates to the use of SLUG as a therapeutic and diagnostic target for these pathologies. The invention also relates to transgenic non-human animals that express SLUG in a regulated fashion.

### BACKGROUND OF THE INVENTION

Obesity represents a major public health problem because of its implications for health. Being overweight or obese increases the risk of many diseases and related conditions. A better knowledge of the molecular mechanisms that control adipose tissue development and function is therefore an important goal for understanding the causes, prevention, and treatment of obesity.

Previous studies have identified a number of transcription factors that are involved in adipocyte differentiation. These include PPARγ and members of the C/EBP family of transcription factors (Morrison and Farmer, 2000; Rosen et al., 2000). While many of the components of the gene regulatory network that controls the differentiation of adipocytes have been elucidated in studies of cultured 3T3-L1 preadipocytes and primary mouse embryonic fibroblasts (MEFs), recent evidence has suggested that additional factors are likely to be necessary in vivo (Soukas et al., 2001; Chen et al., 2005).

While many of the components of the gene regulatory network that control differentiation of adipocytes have been elucidated in studies of cultures 3T3-L1, little is known about the developmental signals that control the development of adipocytes in vivo. The present study establishes for the first time the important role played by SLUG in adipogenesis in vivo and in vitro.

### SUMMARY OF THE INVENTION

According to a first aspect of the invention, there is provided a method of treating a disorder associated with increased or decreased fat storage in a mammal comprising modulating the activity or level of the SLUG protein or SLUG gene in the mammal. This activity or level may either be increased or decreased.

In this study, the inventors have found that SLUG is expressed in human white adipose tissue (WAT). SLUG expression was identified in human subcutaneous adipose tissues isolated from donors with different body mass index (BMI). Surprisingly, the inventors found that SLUG expression was higher in donors with higher BMI. This observation was confirmed by quantitative real time PCR and indicates that expression of SLUG is a common finding in both human and mouse WAT, suggesting a role for SLUG in WAT development.

The inventors then characterised the function of SLUG in WAT development by examining the expression of SLUG during adipocyte differentiation. 3T3-L1 preadipocytes are a well-characterised in vitro model of adipocyte differentiation that can differentiate into mature adipocytes upon exposure to a mixed hormonal stimulus. SLUG expression was shown to be very high before hormonally-induced differentiation of 3T3-L1 preadipocytes and the amount of SLUG mRNA and protein decreased during this hormonal stimulation, suggesting that that SLUG is tightly controlled temporally and spatially during the differentiation of preadipocytes, further suggesting that SLUG is required for adipogenesis.

The zinc-finger transcription factor SLUG (also referred to as SNAI2) is known as an important regulator of normal and tumour development (Sefton et al., 1998; Sanchez-Martin et al., 2004). SLUG controls key aspects of stem cell function, suggesting that similar mechanisms may control normal development and cancer stem cell properties (Inoue et al., 2002; Perez-Losada et al., 2002; Perez-Losada et al., 2003; Perez-Mancera et al., 2005). The post-natal expression of SLUG (SNAI2) and the effects of SLUG deletion and overexpression have been shown to be similar in the mouse and human (Cohen et al., 1998; Perez-Losada et al., 2002; Sánchez-Martin et al, 2002; Oram et al., 2003; Sánchez-Martin et al., 2003; Perez-Mancera et al., 2005; Perez-Mancera et al., 2006). Recent studies showed that SLUG is tightly controlled temporally and spatially in a number of sites including the neural crest and hematopoietic system (Inoue et al, 2002; Perez-Losada et al., 2002). In the major adult tissues, it has been shown that transcripts of the SLUG gene are present in white adipose tissue (WAT) in mice (Perez-Mancera et al., 2005), but no role in the differentiation of this tissue has to date been elucidated.

The inventors analysed SLUG-deficient mice to determine the effect of SLUG expression in WAT development. SLUG-deficient mice were shown to carry much less WAT mass than wild-type mice, showing that SLUG also plays a role in WAT development in vivo. Furthermore, SLUG-deficient mice were found to be protected against obesity induced by a high-fat diet. To confirm that the decrease in WAT mass in SLUG-deficient mice was caused by the absence of SLUG, SLUG-deficient mice were crossed with mice carrying a tetracycline repressible SLUG transgene (Combi-SLUG) that express the transgenic SLUG in WAT tissue. The WAT phenotype was found to be rescued in SLUG-deficient mice by expressing SLUG.

In agreement with these results, Combi-SLUG mice exhibit a strikingly increased WAT mass, supporting the hypothesis that SLUG expression modulates adipose tissue size. Thus, it seems likely that failure to regulate SLUG expression explains why Combi-SLUG mice develop obesity. The inventors further showed that the abolition of SLUG overexpression reversed the WAT alterations induced by SLUG.

Consistent with these in vivo data, SLUG-deficient MEFs showed a dramatically reduced capacity for adipogenesis in vitro compared with wild-type MEFs. In contrast, there was extensive lipid accumulation in Combi-SLUG MEFs.

The molecular mechanism by which SLUG controls WAT development was analyzed and it was found that PPARγ2 expression is altered both in vivo in WAT of SLUG-deficient and Combi-SLUG mice and in vitro in SLUG-deficient MEFs and Combi-SLUG MEFs during the course of adipocytic differentiation. Taken together, these results suggest that SLUG modulates WAT development by affecting PPARγ2 expression. Complementation studies in SLUG-deficient MEFs confirmed this regulation, although Slug was not able to activate transcription from a reporter vector containing the PPARγ2 promoter. When the histone acetylation status in WAT of Combi-Slug and Slug-deficient mice was measured, a correlation between Slug gene expression and histone acetylation status in adipose tissue was identified. This observation is aligned with recent work implicating histone deacetylases HDAC as a mediator of gene regulation modulated by Slug (Peinado et al., 2004; Bermejo-Rodríguez et al., 2006) and prompted the inventors to explore whether Slug is indeed recruited at the PPARγ2 gene promoter. The chromatin precipitation (ChIP) experiments disclosed herein showed that Slug and HDAC1 are bound to the endogenous PPARγ2 promoter in intact chromatin in WAT, and identified a differential HDAC recruitment to the PPARγ2 promoter in a tissue- and Slug-dependent manner. In agreement with these observations, the ChIP analysis confirmed a differential H3 acetylation at the PPARγ2 promoter in a Slug-dependent manner.

Although the Applicant does not wish to be bound by this theory, it is postulated that the most straightforward model for the Slug requirement for PPARγ2 gene expression is that lack of Slug binding to the PPARγ2 gene results in the formation of a silencing complex that represses the expression of the gene, by histone deacetylation. This may have clinical relevance, as HDAC inhibitors are drugs that have activity at doses that are well tolerated by patients in clinical trials (Marks and Jiang, 2005). In agreement with this model, it has been shown that down-regulation of histone deacetylases stimulates adipocyte differentiation (Yoo et al., 2006). Therefore, HDAC inhibitors may be used in the treatment of disorders associated with decreased fat storage.

Therefore, in a further aspect of the invention there is provided a method of treating a disorder associated with decreased fat storage in a mammal comprising modulating the level of transcription from the PPARγ2 gene. Preferably the modulation from the PPARγ2 gene is achieved using a HDAC inhibitor. There are a large number of HDAC inhibitors known in the art, as the skilled reader will appreciate. Examples include short-chain fatty acids such as butyrate and phenylbutyrate, valproate; hydroxamic acids, such as the trichostatins, SAHA and its derivatives, oxamflatin, ABHA, scriptaid, pyroxamide, propenamides; the epoxyketone-containing cyclic tetrapeptides, such as the trapoxins, HC-toxin, chlamydocin, diheteropeptin, WF-3161, Cyl-1 and Cyl-2; the non-epoxyketone-containing cyclic tetrapeptides, such as FR901228, apicidin, the cyclic-hydroxamic-acid-containing peptides (CHAPs); the benzamides, including MS-275 (MS-27-275), CI-994 and other benzamide analogs, depudecin and organosulfur compounds. Preferably the HDAC inhibitor is selected from the group comprising: APHA Compound 8, Apicidin, Sodium Butyrate, (−)-Depudecin, Scriptaid, Sirtinol, and Trichostatin A.

These various results provide evidence that SLUG is a key regulator of adipocyte differentiation both in vivo and in vitro, and indicate that the loss of tight control of SLUG expression can induce obesity and/or lipodystrophy in mice. Therefore, the total or partial repression of SLUG gene expression or of SLUG gene activity is likely to be useful for treating or preventing any disorder associated with fat storage. In particular, such conditions include obesity, anorexia and lipodystrophies. In view of the demonstration herein that SLUG is also expressed in human white fat, this provides a very important lead to the development of targeted drugs for treatment of these pathologies in humans. In particular, for disorders associated with a decrease in fat storage, e.g. anorexia, it is desirable to treat a patient suffering from the disorder by increasing the expression from the SLUG gene in the patient, administering SLUG to the patient, or administering a compound which acts as an agonist of SLUG activity to the patient. Conversely, for disorders associated with an increase in fat storage, e.g. obesity, it is desirable to treat a patient suffering from the disorder by decreasing the expression from the SLUG gene in the patient, or administering a compound which acts as an antagonist to SLUG activity to the patient.

In one embodiment of the first aspect of the invention the method comprises administering the SLUG protein, or a functional equivalent of the SLUG protein such as a SLUG mutant or a modified form of the SLUG protein to the mammal. The functional equivalent of the SLUG protein may show either an increase or a decrease in one or more of the activities possessed by the wild type SLUG protein.

The terms “SLUG polypeptide” and “SLUG protein” refer to a member of the SLUG family of zinc-finger transcription factors which is an important regulator of normal and tumour growth. SLUG controls key aspects of stem cell function. The amino acid sequence of the human SLUG protein is known (see, for example, NCBI, Accession number AAB58705).

The term “SLUG gene” refers to the gene coding for the SLUG protein. The nucleotide sequence of the human SLUG gene is known (see, for example, NCBI, Accession number U97060) and this is a preferred gene for use in aspects of the invention referred to herein.

The term “activity” when used in relation to the SLUG protein refers to any activity possessed by the wild type protein. Such activities include the protein's ability to bind specifically to DNA at particular sequence defined consensus sites as well as its ability to induce transcription from such DNA. Such DNA sequences include known DNA promoter sequences. In a preferred embodiment of this aspect of the invention the SLUG protein binds to and induces transcription from the E-cadherin promoter. The invention, therefore, envisages using analytically-detectable proteins placed under the control of the E-cadherin promoter to identify and test SLUG protein agonists and antagonists. Preferred proteins include, but are not limited to, luciferase and green fluorescent protein

Further activities also include the SLUG protein's ability to bind to other proteins. In particular, in the context of the present invention, the term “activity” also refers to the protein's ability to induce adipogenesis and, therefore, to its ability increase the amount of adipose tissue present in a mammal. The term “adipogenesis” refers to the formation of fat or fatty tissue. It also refers to the development of fat precursor cells into mature white or brown adipose tissue.

A protein showing a decrease in one or more of the activities possessed by the normal SLUG protein may be useful for inhibiting the action of normal SLUG. For example, such a protein may retain the ability to bind to DNA, but may lose the ability to activate transcription from said DNA. Therefore, such a protein could act as a competitive inhibitor for normal SLUG.

The term “functional equivalent”, as used herein, refers to a protein sequence that has an analogous function to the sequence of which it is a functional equivalent. By “analogous function” is meant that the sequences share a common function, for example, in the regulation of adipogenesis, and, in some embodiments, a common evolutionary origin. The term “functional equivalent” is intended to include all fragments, mutants, hybrids, variants, analogs, or chemical derivatives of a molecule.

In some embodiments, a functionally equivalent sequence may exhibit sequence identity with the sequence of which it is a functional equivalent. Preferably, the sequence identity between the functional equivalent and the sequence of which it is a functional equivalent is at least 50% across the length of the functional equivalent. More preferably, the identity is at least 60% across the length of the functional equivalent. Even more preferably, identity is greater than 70%, 80%, 85%, 90%, 95%, 96%, 97%, 98% or 99% across the length of the functional equivalent.

Functional equivalents include mutants of the sequences of which they are functional equivalents, i.e. containing amino acid substitutions, insertions or deletions from said sequence, provided that function is retained. Functional equivalents with improved function compared to the sequences of which they are functional equivalents may be designed through the systematic or directed mutation of specific residues in said sequences. Functional equivalents include sequences containing conservative amino acid substitutions that do not affect the function or activity of the sequence in an adverse manner. Particularly preferred mutants are those in which at least 1, preferably 2, 3, 4, 5, 6, 7, 8, 9 or 10 amino acids have been altered from the wild type SLUG sequence.

Functional equivalents include fragments of the SLUG protein. For example, the SLUG protein may be truncated at one or both termini so as to retain functional domains that are important for its activity. Such fragments may be truncated, for example, by between 10 and 30 amino acids, between 15 and 25 amino acids, or around 20 amino acids, at either one or both the N terminus and C terminus.

The SLUG protein or functional equivalent can work either as an isolated peptide or as a fusion with another entity. Any peptide used in the context of the present invention will typically be a polypeptide e.g. consisting of between 10 and 500 amino acids. The polypeptide preferably consists of no more than 200 amino acids (e.g. no more than 190, 180, 170, 160, 150, 140, 130, 120, 110, 100, 90, 80, 70, 60, 50, 45, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or no more than 10). Details of particular preferred polypeptides for use in accordance with the invention are given below.

Partner entities for fusion proteins as mentioned above include, for example, functional entities that will impart additional functionality to the SLUG component of the molecule, including Fc domains, drug moieties, components to impart additional stability to the molecule, targeting domains such as antibodies or fragments thereof and so on. Other examples will be clear to those of skill in the art.

The term “transcription product of SLUG gene” refers to the mRNA of SLUG gene.

The term “translation product of SLUG gene” refers to the SLUG protein. Again, the human SLUG protein is preferred.

In a further preferred embodiment of the first aspect the method comprises administering a compound that modulates the activity of the SLUG protein, or modulates transcription and/or translation of the SLUG gene to the mammal. The compound may upregulate the activity of the SLUG protein or upregulate the transcription and/or translation of the SLUG gene; or the compound may downregulate the activity of the SLUG protein or downregulate the transcription and/or translation of the SLUG gene.

The invention contemplates the use of any compounds which are capable of either modulating the activity of the SLUG protein, or modulating transcription and/or translation of the SLUG gene in the methods of the invention. Compounds which are known to modulate transcription or translation of the SLUG gene include, for example, antisense SLUG mRNA, ribozymes, triple helix molecules, small interference RNA (siRNA), BCR-ABL protein, c-Kit protein, FGF1 protein, VEGF165, SCF, ngn3 protein, FKHR protein, PAX3 and beta catenin. Compounds which modulate the activity of the SLUG protein include, for example, anti-SLUG antibodies. All of these entities, and those with similar function, can be used in the prevention, treatment and/or diagnosis of disease conditions that are listed herein. Furthermore, functional equivalents of these compounds, including for example, peptides, specific protein domains, fusion proteins and so on, will be of similar utility in the present invention. Those passages of this specification relating to functional equivalents of the SLUG protein are intended to be of similar applicability to the compounds listed above.

The term “modulates” refers to both upregulation and downregulation of one or more of the normal activities of the SLUG protein.

In a further preferred embodiment of the first aspect of the invention, the disorder associated with increased or decreased fat storage in a mammal may be any one of, but not limited to, obesity, anorexia or lipodystrophy.

The term “obesity” refers to any condition in which the natural energy reserve, stored in the fatty tissue of mammals, in particular humans, is increased to a point where it is a risk factor for certain health conditions or increased mortality. Obesity is typically evaluated by measuring BMI (body mass index) in combination with waist circumference. Excessive body weight has been shown to correlate with various diseases, particularly cardiovascular disease, diabetes mellitus type 2, sleep apnea, and osteoarthritis. Therefore, as envisaged by the invention “treating obesity” refers to treating any condition known to be associated with obesity. Compounds for the treatment of obesity according to the invention may be co-administered with other moieties that are used for the treatment of obesity, including one or more appetite suppressants such as, for example, phentermine and sibutramine; lipase inhibitors such as orlistat; anti-depressants such as bupropion; and other trial drugs such as rimonabant and ciliary neurotrophic factor.

The term “lipodystrophy” refers to any conditions characterised by a disturbance of lipid (fat) metabolism that involves the partial or total absence of fat and the abnormal deposition and distribution of fat in the body. The term also includes the more specific term “lipoatrophy” which is used when describing the loss of fat from one area (e.g. the face). Lipodystropies can be a possible side effect of HIV medication (mainly the use of protease inhibitors). Other lipodystropies manifest as the excess or lack of fat in various regions of the body. These include but are not limited to having sunken cheeks, “humps” on the back or back of the neck and small lumps or dents in the skin formed by repetitive injections in the same spot (e.g. insulin use in diabetics). Lipodystrophy can also be caused by metabolic abnormalities due to genetic issues. These are often characterised by insulin resistance. Compounds according to the invention for the treatment of lipodystrophy may be co-administered with other moieties that are used for such treatment, including, for example, poly-L-lactic acid (e.g. Sculptra).

The term “anorexia” refers to any eating disorder characterised by markedly reduced appetite or total aversion to food. The term also includes “anorexia nervosa”. Compounds according to the invention for the treatment of anorexia nervosa may be co-administered with other moieties that are used in these treatments, including cyproheptadine

In a second aspect the invention relates to the use of the SLUG protein, or a functional equivalent of the SLUG protein for treating or preventing a disorder associated with increased or decreased fat storage in a mammal. As mentioned above, a functional equivalent of the SLUG protein may show either an increase or a decrease in one or more of the activities possessed by the normal SLUG protein.

In a third aspect the invention relates to the use of a compound that modulates the activity of the SLUG protein, or modulates transcription and/or translation of the SLUG gene for treating or preventing a disorder associated with increased or decreased fat storage in a mammal. Such a compound may either upregulate or downregulate the activity of the SLUG protein or the transcription and/or translation of the SLUG gene.

In a preferred embodiment of the second and third aspects of the invention, the disorder associated with increased or decreased fat storage in a mammal may be any one of, but not limited to, obesity, anorexia or lipodystrophy.

In a fourth aspect the invention relates to a method for screening for a compound that modulates the fat-related activity of the SLUG protein or the level of transcription or translation of the SLUG gene, comprising administering a candidate compound to a test non-human mammal and monitoring the effect on fat storage in that mammal.

In a preferred embodiment of the fourth aspect of the invention monitoring the effect on fat storage in the mammal comprises assessing the amount of adipose tissue. Preferably, the method includes comparing the amount of adipose tissue in a first test non-human animal with the amount of adipose tissue in a second test non-human mammal of the same species. More preferably, the method includes a comparison of the amount of adipose tissue in the first test non-human animal with the amount of adipose tissue in a second test mammal of the same species, where the second test mammal has been administered a placebo. More preferably, the method includes a comparison of the amount of adipose tissue in the first test non-human animal before and after administration of the candidate compound. More preferably the adipose tissue is white adipose tissue. Methods for the assessment of the amount of adipose tissue in a mammal will be clear to those of skill in the art and specifically include those referred to herein. For example, in the context of adipocyte differentiation, the use of 3T3-L1 preadipocytes forms a well-characterized in vitro model of adipocyte differentiation that can differentiate into mature adipocytes upon exposure to a mixture of hormonal stimuli (Ntambi et al, 1988).

In a further preferred embodiment of the fourth aspect of the invention the first and/or second test non-human mammal of the method is a transgenic or knockout non-human mammal that has been transformed to express higher, lower or absent levels of a SLUG polypeptide. Preferably the transgenic or knockout mammal comprises in its genome a transgene that comprises a nucleic acid sequence encoding the SLUG protein, wherein the expression of the transgene can be regulated exogenously by an effector substance. The expression of the transgene may, for example, be tetracycline-regulated. More preferably, the transgenic or knockout mammal suffers from a disorder associated with increased or decreased fat storage. More preferably, the transgenic or knockout non-human mammal suffers from obesity, anorexia or lipodystrophy.

The expression “non-human mammal”, as used herein, includes any non-human animal belonging to the class of mammals. The non-human mammal is preferably a mouse but may be another mammalian species, for example another rodent, for instance a rat, hamster or a guinea pig, or another species such as a monkey, pig, rabbit, or a canine or feline, or an ungulate species such as ovine, caprine, equine, bovine, or a non-mammalian animal species. In a particular embodiment, the transgenic or knockout non-human animal provided by the invention is a murine animal. The term “murine” includes mice, rats, guinea pigs, hamsters and the like. In a preferred embodiment the murine animal is a rat or a mouse; most preferably the non-human mammal of the invention is a mouse.

Although the use of transgenic animals poses questions of an ethical nature, the benefit to man from studies of the types described herein is considered vastly to outweigh any suffering that might be imposed in the creation and testing of transgenic animals. As will be evident to those of skill in the art, drug therapies require animal testing before clinical trials can commence in humans and under current regulations and with currently available model systems, animal testing cannot be dispensed with. Any new drug must be tested on at least two different species of live mammal, one of which must be a large non-rodent. Experts consider that new classes of drugs now in development that act in very specific ways in the body may lead to more animals being used in future years, and to the use of more primates. For example, as science seeks to tackle the neurological diseases afflicting a ‘greying population’, it is considered that we will need a steady supply of monkeys on which to test the safety and effectiveness of the next-generation pills. Accordingly, the benefit to man from transgenic models such as those described herein is not in any limited to mice, or to rodents generally, but encompasses other mammals including primates. The specific way in which these novel drugs will work means that primates may be the only animals suitable for experimentation because their brain architecture is very similar to our own.

This aspect of the invention aims to reduce the extent of attrition in drug discovery and development. Whenever a drug fails at a late stage in testing, all of the animal experiments will in a sense have been wasted. Stopping drugs failing therefore saves test animals' lives. Therefore, although the present invention relates to transgenic animals, the use of such animals should reduce the number of animals that must be used in drug testing programmes and decrease attrition rates in clinical assays in humans.

In a fifth aspect the invention relates to a method for screening for a compound that modulates the activity of the SLUG protein or a functional equivalent of the SLUG protein or modulates the level of transcription or translation of the SLUG gene, comprising contacting a cell with a candidate compound and monitoring the effect on the amount of lipid accumulation in the cell. The cell may initially (i.e. before the cell is contacted with the candidate compound) express altered levels of SLUG in comparison to a wild type cell.

In an alternative methodology, in which transcription of the SLUG gene is to be targeted by the compound, the screening method may employ a cell or animal which expresses a synthetic construct comprising the SLUG promoter linked to a reporter molecule. The reporter molecule can then be used to assay for the efficacy of the compound in reducing SLUG expression. Suitable reporter molecules will be clear to those of skill in the art and include assayable enzymes such as 13-galactosidase and alkaline phosphatase, marker proteins, such as Green Fluorescent Protein (GFP), and labels such as radioactive isotopes.

In a preferred embodiment of the fifth aspect of the invention, the cell is derived from a transgenic or knockout non-human mammal as described above in relation to the first aspect of the invention. The cell may be an embryonic fibroblast cell, for example, derived from a transgenic or knockout non-human mammal as described above in relation to the first aspect of the invention. More preferably the cell is a mouse or human embryonic fibroblast (MEF or HEF) cell. Alternatively, the cell may be transfected with a gene encoding SLUG or a functional equivalent thereof.

In a preferred embodiment of the fifth aspect of the invention, monitoring the effect on the cell optionally comprises monitoring the level or activity of PPARγ2 in the cell. The results disclosed herein suggest that SLUG may modulate WAT development by affecting PPARγ2 expression. The expression of PPARγ2 was decreased in the WAT of SLUG-deficient mice and increased in the WAT of Combi-SLUG mice. A lower level of PPARγ2 expression or activity in one of the assays described above is thus reflective of lowered SLUG expression or activity.

In a sixth aspect the invention relates to a compound that modulates the activity of the SLUG protein or a functional equivalent of the SLUG protein or modulates the level of transcription or translation of the SLUG gene, obtained or obtainable by any of the methods the fourth and fifth aspects of the invention.

In a seventh aspect the invention relates to a pharmaceutical composition comprising a protein as defined in the second aspect of the invention, a compound as defined in the third aspect of the invention or a compound according to the sixth aspect of the invention.

In an eighth aspect the invention relates to a compound according to the sixth aspect of the invention for use as a medicament.

In a ninth aspect the invention relates to the use of a compound according to the sixth aspect of the invention in the manufacture of a medicament for treating or preventing a disorder associated with increased or decreased fat storage. Preferably the disorder is obesity, anorexia or lipodystrophy.

In a tenth aspect the invention relates to a method for altering fat storage in a mammal comprising administering a protein according to the second aspect of the invention or a compound according to the third or sixth aspects of the invention or a composition according to the seventh aspect of the invention to the mammal.

### DEFINITIONS

In order to facilitate the understanding of the instant description, the meaning of some terms and expressions in the context of the invention are explained below.

The term “gene” refers to a molecular chain of deoxyribonucleotides encoding a protein.

The term “DNA” refers to deoxyribonucleic acid. A DNA sequence is a deoxyribonucleotide sequence.

The term “cDNA” refers to a nucleotide sequence complementary of a mRNA sequence.

The term “RNA” refers to ribonucleic acid. An RNA sequence is a ribonucleotide sequence.

The term “mRNA” refers to messenger ribonucleic acid, which is the fraction of total RNA which is translated into proteins.

The term “protein” refers to a molecular chain of amino acids with biological activity.

The term “antibody” refers to a glycoprotein exhibiting specific binding activity to a particular protein, which is called “antigen”. The term “antibody” comprises monoclonal antibodies, polyclonal antibodies, either intact or fragments thereof, recombinant antibodies, etc., and includes human, humanised and non-human origin antibodies. “Monoclonal antibodies” are homogenous populations of highly specific antibodies directed against a single site or antigenic “determinant”. “Polyclonal antibodies” include heterogeneous populations of antibodies directed against different antigenic determinants.

The term “epitope”, as it is used in the present invention, refers to an antigenic determinant of a protein, which is the amino acid sequence of the protein recognised by a specific antibody.

The following examples illustrate the invention and should not be considered to limit the scope thereof. The practice of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al., Molecular Cloning, A Laboratory Manual (2001) Cold Harbor-Laboratory Press, Cold Spring Harbor, N.Y. or Ausubel et al., Current protocols in molecular biology (1990) John Wiley and Sons.

Unless defined otherwise, all technical and scientific terms used herein have the same meaning as commonly understood by one of ordinary skill in the art to which this invention belongs.

All documents cited herein are hereby incorporated by reference.

### BRIEF DESCRIPTION OF THE FIGURES

FIG. 1. Expression of both human and mouse Slug was analyzed by RT-PCR. 36B4 were used to check cDNA integrity and loading. A) Slug expression in mouse WAT. Expression of Combi-Slug, endogenous Slug, and adipocyte fatty acid-binding protein (aP2) was analyzed by RT-PCR in WAT derived of Combi-Slug, Slug-deficient mice and control mice. The PCR products were transferred to a nylon membrane and analyzed by hybridization with a specific probe. B) SLUG expression in human tissues. Expression of endogenous SLUG was analyzed by RT-PCR in a variety of human tissues like liver (1), heart (2), hWAT#1 (3), kidney (4), spleen (5) and no cDNA (6). C) SLUG expression in human adipose tissue. Expression of endogenous SLUG, and adipocyte fatty acid-binding protein (aP2) was analyzed by RT-PCR in human subcutaneous adipose tissue RNA (Zen-bio, Inc) corresponding to donors with different BMI. Human peripheral blood as a tissue where human SLUG is not expressed (lane 1), human subcutaneous adipose tissue coming from a donor with a BMI=21.23 (hWAT#1, lane 2), human subcutaneous adipose tissue coming from a donor with a BMI=27.37 (hWAT#2, lane 3), human subcutaneous adipose tissue coming from a donor with a BMI=32.55 (hWAT#3, lane 4), and no cDNA (lane 5). D) Quantification of SLUG (SNAI2) expression by real-time PCR in human adipose tissues. Percentage of SLUG transcripts with reference to β-actin is shown in hWAT#1, hWAT#2, and hWAT#3 human adipose tissue samples. Values are means±SEM of three independent experiments. Differences between” hWAT#1 and hWAT#3″ were statistically significant (P<0.05) as determined by Mann-Whitney's test. However, each hWAT sample comes from only one individual and the apparent correlation between the BMI and SLUG expression may depend on population variation. E) Slug expression in brown adipose tissue (BAT). Expression of mouse Slug was analyzed by RT-PCR in BAT derived of control mice.

FIG. 2. Time course of the expression of SLUG during differentiation of preadipocytes. 3T3-L1 cells incubated for the indicated times after the onset of exposure to inducers of differentiation were subjected to Northern blot analysis (A), or to immunoblot analysis (B). After exposure to a hormonal cocktail, CEBPβ is actively expressed and then begins to diminish around day 2 of hormonal induction, at which point the expression of C/EBPα and PPARγ increase (23). C/EBPα and PPARγ induce programs of gene expression leading to the differentiation of mature adipocytes (2, 17, 24). It has been documented that selective disruption of PPARγ2 impairs the development of adipose tissue and is absolutely required for differentiation (16), while C/EBPα is not strictly required for adipogenesis (25). These data are representative of three independent experiments. These data are representative of three experiments. C) Time course of Slug expression during adipocyte differentiation in control and Combi-Slug MEFs. MEFs cells incubated for the indicated times after the onset of exposure to inducers of differentiation were subjected to immunoblot analysis.

FIG. 3. Comparison of WAT samples in SLUG-deficient, Combi-SLUG and control mice. A) A ventral view of SLUG-deficient, Combi-SLUG and control mice (upper row). B) A comparison of reproductive fat pads of SLUG-deficient, Combi-SLUG and control mice (second row). C) Hematoxylin/eosin stained sections of reproductive fat pads from male SLUG-deficient, Combi-SLUG and control mice (third row -20×-, and fourth row -40×-).

FIG. 4. A comparison of BAT samples in SLUG-deficient, Combi-SLUG and control mice. Hematoxylin/eosin stained sections of interescapular brown fat from SLUG-deficient, Combi-SLUG and control mice (20×).

FIG. 5. Adipocytic accumulation in Combi-SLUG mice. A) Hematoxylin-eosin stained sections of the liver and kidney tissues coming from wild-type and Combi-SLUG mice. B) Tumour and histologic appearance of a lipoma developed in CombiTA-SLUG mice after Hematoxylin-eosin staining. FIG. 6. WAT size in CombiTA-SLUG mice after suppression of SLUG expression by tetracycline treatment. A) Analysis of tetracycline-dependent SLUG expression in inguinal fatpad for CombiTA-SLUG (−tet, +tet in water) by RT-PCR. 36B4 was used to check cDNA integrity and loading. B) WAT weights in CombiTA-SLUG mice after suppression of SLUG expression by tetracycline treatment (4 gr/L) for 3 weeks. Differences were statistically significant (P<0.01) as determined by Mann-Whitney's test. WAT mass was not afected in control mice under tetracycline treatment (4 gr/L) for 3 weeks.

FIG. 7. Adipogenic gene expression in SLUG-deficient and Combi-SLUG WAT. Western blot analyses of gene expression in WAT of SLUG-deficient mice, control mice and Combi-SLUG mice. B) Quantification of PPARγ2 expression by real-time PCR in WAT of Slug-deficient mice, control mice and Combi-Slug mice (n=3). Percentage of PPARγ2 transcripts with reference to b-actin is shown. Differences were statistically significant (P<0.03) as determined by Mann-Whitney's test. C) Western blot analyses of fat cell markers such glut4, adiponectin, adipsin, and apt in WAT of Slug-deficient mice, control mice and Combi-Slug mice. These data are representative of three independent experiments.

FIG. 8. Altered lipid accumulation in SLUG-deficient and Combi-SLUG MEFS. A) Western blot analyses of SLUG expression in control, SLUG-deficient and Combi-SLUG MEFs before exposure to inducers of differentiation. Actin was used to check protein loading. B) Primary embryonic fibroblasts from each line were cultured in the presence of standard differentiation induction medium. At day 8 after induction of adipocyte differentiation, cells were fixed and stained for neutral lipids with Oil Red O. The original magnification is ×20. This experiment was repeated three times using cells prepared from all lines and from different embryos and similar results were obtained.

FIG. 9. Adipogenic gene expression in SLUG-deficient and Combi-SLUG MEFs during differentiation. A) Western-blot analysis of adipogenic genes at day 8 post-induction in SLUG-deficient, control and Combi-SLUG MEFs. 3T3-L1 cells at day 2 post-induction are used as a control. B) The pattern of adipogenic gene expression in Combi-Slug MEFs is similar to a terminally differentiated cell at day 8 post-induction. However, the pattern of adipogenic gene expression in Slug-deficient MEFs at day 8 post-induction is similar to 3T3-L1 cells at day 2 post-induction. Quantification of PPARγ2 expression by real-time PCR in control, Slug-deficient and combi-Slug±DOX MEFs at day 0, 2, 4 and 8 post-induction. A representative ethidium bromide agarose gel is shown close to the percentage of PPARγ2 transcripts with reference to b-actin is shown. Differences were statistically significant (P<0.01) as determined by Mann-Whitney's test. C) Expression of ap2 was studied by western-blot in control and Combi-Slug MEFs at day 0, 2, 4 and 8 post-induction. D) Slug −/− MEFs were induced with differentiation medium in the presence or absence of 10 μM PPARγ ligand troglitazone. At day 0, 2, 4 and 8 post-induction ap2 protein expression was studied by western-blot. Actin was included as a loading control. E) At day 8 post-induction cells were stained for droplets with Oil Red 0 and the morphological differentiation of Slug-deficient MEFs+troglitazone is shown. These data are representative of three independent experiments.

FIG. 10. Retrovirus-mediated overexpression of SLUG rescues the impaired in vitro adipogenesis of SLUG-deficient MEFs. A) Western blot analysis of SLUG and PPARγ2 protein in SLUG −/− MEFs infected with either a control retroviral vector or one expressing SLUG (pQCXIP-mSLUG) at day 0, 2, 4, and 8 after exposure to inducers of adipocyte differentiation. Actin was included as a loading control. B) Quantification of PPARγ2 expression by real-time PCR in Slug −/− MEFs infected with either a control retroviral vector or one expressing Slug (pQCXIP-mSlug) at day 0, 2, 4 and 8 post-induction. A representative ethidium bromide agarose gel is shown close to the percentage of PPARγ2 transcripts with reference to b-actin is shown. Differences were statistically significant (P<0.01) as determined by Mann-Whitney's test. C) Analysis of ap2 protein by western blot in Slug −/− MEFs infected with either a control retroviral vector or one expressing Slug (pQCXIP-mSlug) at day 0, 2, 4, and 8 after exposure to inducers of adipocyte differentiation. Actin was included as a loading control. D) At day 8 after induction of adipocyte differentiation, cells were observed by light microscopy with Oil-Red-O staining (the original magnification is ×20). These data are representative of three independent experiments.

FIG. 11. Slug does not transactivate the PPARg2 promoter. A 1 kb proximal promoter region of human PPARγ2 was previously shown to be sufficient to drive the PPARγ2′ s expression in reporter assays (Fajas et al., 1997) and it is active in U2OS cells when co-transfected with C/EBPα and C/EBPβ expression vectors. To directly assess the ability of Slug to activate transcription from DNA sequences present in the PPARγ2 promoter, an expression vector containing a Slug cDNA was co-transfected into U2OS cells along with the reporter vector containing the PPARγ2 promoter (pGL3-hPPARg2p1000 vector). Luciferase reporter assays demonstrate lack of responsiveness of the human PPARg2 reporter to Slug. The number shown at the left of the reporter construct denotes the 5′-boundaries (bp upstream of the initiation site). These data are representative of three independent experiments.

FIG. 12. Histone acetylation status. Protein acetylation patterns of different tissue surgical samples removed from different wild-type, Slug-deficient and Combi-Slug mice. Data shows high increase in histone H3 acetylation in Combi-Slug WAT and decrease in histone H3 acetylation in Slug −/− WAT compare with wt mice. Brain and liver were used as negative upregulation profile. Samples were blotted with anti-acetyl histone H3 (Upstate Biotechnology, Lake Placid, N.Y.). Wild type tissue from a Histone Deacetylase inhibitor (HDACi) treated mouse was used as a positive upregulation and Acetylated H3-increased sample. Core H3Coomassie stained was used as loading control. These data are representative of three independent experiments.

FIG. 13. Recruitment analysis of HDAC, SLUG and c/EBPα to mouse PPARγ2 gene promoter. A) Schematic depiction of the mouse PPARγ2 promoter sequence from −1205 to −46 (GenBank: AY243584), with arrows indicating the forward and reverse primers used to amplify ChIP products. Pairs of primers 1 and 2 are around two Slug DNA-binding sites, and pairs of primers 3 are not around Slug DNA-binding sites. B) WAT and Liver chromatin immunoprecipitation from different mice (wt, Combi-SLUG and SLUG −/−) using polyclonal anti-HDAC1 (H-51), anti-SLUG (H-140) or anti-c/EBPα (14AA) from Santa Cruz Biotechnology Inc., (Santa Cruz, Calif., USA). Data shows a differential HDAC recruitment to the PPARγ2 promoter in a tissue- and genetic background-dependent manner. The presence of the promoter DNA before immunoprecipitation was confirmed by PCR (Input). C/EBPa was used as a positive response element from PPARγ2 gene promoter. PCR products were resolved in 2% agarose gels containing ethidium bromide. C) WAT chromatin immunoprecipitation from different mice (wt, Combi-SLUG and SLUG −/−) using polyclonal anti-HDAC1 (H-51), or anti-SLUG (H-140) from Santa Cruz Biotechnology Inc., (Santa Cruz, Calif., USA). Data shows no Slug recruitment to the PPARγ2 promoter using pairs of primers that are not around Slug DNA-binding sites. The presence of the promoter DNA before immunoprecipitation was confirmed by PCR (Input). PCR products were resolved in 2% agarose gels containing ethidium bromide. D) WAT chromatin immunoprecipitation from different mice (wt, Combi-SLUG and SLUG −/−) using anti-acetyl histone H3 (Upstate Biotechnology, Lake Placid, N.Y.) or anti-SLUG (H-140) from Santa Cruz Biotechnology Inc., (Santa Cruz, Calif., USA). Data shows a correlation between Slug expression and H3 acetylation at the PPARγ2 promoter. The presence of the promoter DNA before immunoprecipitation was confirmed by PCR (Input). PCR products were resolved in 2% agarose gels containing ethidium bromide. E) C/EBPα and C/EBPβ ability to transactivate the PPARg2 promoter in Slug-deficient cells. To directly assess the ability of C/EBPα and C/EBPβ to activate transcription from DNA sequences present in the PPARγ2 promoter in Slug-deficient cells, C/EBPα and C/EBPβ expression vectors were co-transfected into Slug −/− MEF along with the reporter vector containing the PPARγ2 promoter (pGL3-hPPARg2p1000 vector) in the presence (+) and in the absence (−) of Slug. Luciferase reporter assays demonstrate an efficient responsiveness of the human PPARg2 reporter to C/EBPα and C/EBPβ in the presence of Slug. These data are representative of three independent experiments.

FIG. 14. Representative growth curves of control, Combi-Slug and Slug-deficient mice under chow (A) and HFD (B). Body weight was determined once every two weeks (n=8; 4 females and 4 males). Values are means±SEM. Differences between “control and Slug-deficient mice” and “control and Combi-Slug mice” were statistically significant (P<0.05) as determined by Mann-Whitney's test.

### EXAMPLES

### Example 1

### Materials And Methods

### Mice

Animals were housed under non-sterile conditions in a conventional animal facility. SLUG-deficient and Combi-SLUG mice have been previously described (Jiang et al., 1998). Combi-Slug mice are analyzed on a wild-type background unless otherwise indicated. Combi-Slug×Slug −/− mice were generated as follow: Heterozygous SLUG +/− mice were bred to Combi-SLUG transgenic mice to generate compound heterozygotes. F1 animals were crossed to obtain null SLUG −/− mice heterozygous for Combi-SLUG transgenic mice as described (Pérez-Mancera et al., 2005). The animals were maintained regular chow diet unless otherwise indicated. All experiments were done according to the relevant regulatory standards.

### Histological Analysis

All tissue samples were closely examined under the dissecting microscope and processed into paraffin, sectioned and examined histologically. All tissue samples were taken from homogenous and viable portions of the resected sample by the pathologist and fixed within 2-5 min. of excision. Hematoxylin- and eosin-stained sections of each tissue were reviewed by a single pathologist (T.F.). For comparative studies, age-matched mice were used.

### Preparation of Primary Mouse Embryonic Fibroblasts (MEF)

Heterozygous SLUG +/− mice were crossed to obtain wild-type and null SLUG −/− embryos. Primary embryonic fibroblasts were harvested from 13.5 d.p.c. embryos. Head and organs of day 13.5 embryos were dissected; fetal tissue was rinsed in PBS, minced, and rinsed twice in PBS. Fetal tissue was treated with trypsin/EDTA and incubated for 30 min at 37° C. and subsequently dissociated in medium. After removal of large tissue clamps, the remaining cells were plated out in a 175 cm² flask. After 48 h, confluent cultures were frozen down. These cells were considered as being passage 1 MEFs. For continuous culturing, MEF cultures were split 1:3. MEFs and the φNX ecotropic packaging cell line were grown at 37° C. in Dubelcos-modified Eagle's medium (DMEM; Boehringer Ingelheim) supplemented with 10% heat-inactivated FBS (Boehringer Ingelheim). All the cells were negative for mycoplasma (MycoAlert-rM Mycoplasma Detection Kit, Cambrex).

### Adipocyte Differentiation

3T3-L1 preadipocytes were cultured as described (Lin and Lane, 1994). Wild-type, Combi-SLUG and SLUG −/− MEFs were cultured at 37° C. in standard D-MEM:F12 medium (Gibco) supplemented with 10% heat-inactivated FBS (Hyclone), 100 units/ml penicillin (Biowhittaker), and 100 μg/ml streptomycin (Biowhittaker). 10⁶ cells of each genotype were plated to 10 cm plastic dishes and propagated to confluence. Two days after confluence, the adypocite differentiation program was induced by feeding the cells with standard medium supplemented with 0.5 mM 3-isobutyl-1-Methylxantine (Sigma), 1 μM dexamethasone (Sigma) and 5 μg/ml insulin (Sigma) for two days, and then, with standard medium supplemented with 5 μg/ml insulin for 6 days. This medium was renewed every two days. Troglitazone (Calbiochem), or vehicle, was used at 10 μM during the 8 days of differentiation when required. After 8 days, the appearance of cytoplasmic lipid accumulation was observed by Oil-Red-O staining. Lipid accumulation was defined as a percentage of cells that were Oil-Red-O positive by counting ˜700 cells in at least three independent replicates for each experiment. Briefly, cells were washed with phosphate-buffered saline (PBS), and then fixed with 3.7% formaldehyde for 2 minutes. After a wash with water, cells were stained with 60% filtered Oil-Red-O stock solution (0.5 g of Oil-Red-O (Sigma) in 100 ml of isopropanol) for 1 hour at room temperature. Finally, cells were washed twice in water and photographed. To prepare RNA for Northern blotting, and proteins for Western blotting, cells were harvested at days 0, 2, 4 and 8 of differentiation.

### RNA Extraction

Total RNA was isolated in two steps using TRIzol (Life Technologies, Inc., Grand Island, N.Y.) followed by Rneasy Mini-Kit (Qiagen Inc., Valencia, Calif.) purification following the manufacturer's RNA Clean-up protocol with the optional On-column Dnase treatment. The integrity and the quality of RNA was verified by electrophoresis and its concentration measured.

### Reverse Transcription-PCR (RT-PCR)

Human WAT samples were obtained from Zen-bio (hWAT#1 is Cat. Number RNA-T10-1 with a body mass index (BMI): 21.23; hWAT#2 is Cat. Number RNAT10-2 with a BMI: 27.27; hWAT#3 is Cat. Number RNA-T10-3 with a BMI: 32.55). To analyze expression of CombitTA-SLUG and endogenous SLUG in mouse cell lines and mice, RT was performed according to the manufacturer's protocol in a 20-μl reaction containing 50 ng of random hexamers, 3 μg of total RNA, and 200 units of Superscript II RNase H-reverse transcriptase (GIBCO/BRL). The sequences of the specific primers were as follows: CombipolyA-B1: 5′-TTGAGTGCATTCTAGTTGTG-3′; mSLUGF: 5′-GTTTCAGTGCAATTTATGCAA-3′; mSLUGB: 5′-TTATACATACTATTTGGTTG-3′. To analyse expression of human SLUG, the thermocycling parameters for the PCR reactions and the sequences of the specific primers were as follows: 30 cycles at 94° C. for 1 min, 56° C. for 1 min, and 72° C. for 2 min; sense primer 5′-GCCTCCAAAAAGCCAAACTA-3′ and antisense primer 5′-CACAGTGATGGGGCTGTATG-3′. The PCR products were confirmed by hybridization with specific probes. Amplification of apt and 36B4 served as a control to assess the adipose tissue and the quality of each RNA sample, respectively.

### Real-Time PCR Quantification

Real-time quantitative PCR was developed and carried out in human WAT samples obtained from Zen-bio (hWAT#1; hWAT#2, and hWAT#3) for the detection and quantitation of the SLUG expression. The PCRs were set up in a reaction volume of 50 μl using the TaqMan PCR Core Reagent kit (PE Biosystems). PCR primers were synthesized by Isogen. Each reaction contained 5 μl of 10× buffer; 300 nM each amplification primer; 200 μM each dNTP; and 1.25 U AmpliTaq Gold, 2 mM MgCl₂, and 10 ng cDNA. cDNA amplifications were carried out in a 96-well reaction plate format in a PE Applied Biosystems 5700 Sequence Detector. Thermal cycling was initiated with a first denaturation step of 10 min at 95° C. The subsequent thermal profile was 40 cycles of 95° C. for 15 s, 55° C. for 30 s, 72° C. for 1 min. Multiple negative water blanks were tested and a calibration curve determined in parallel with each analysis. Although equal amounts of cDNA were used, a 13-actin endogenous control was included to relate SLUG expression to total cDNA in each sample.

Similarly a real-time quantitative PCR was developed and carried out in control, Slug-1- and Combi-Slug WAT samples for the detection and quantitation of the PPARγ2 expression. Thermocycling was carried out for 40 cycles in triplicate. Each cycle consisted of 94° C. for 15 seconds, 56° C. for 30 seconds and 72° C. for 30 seconds. PPARγ2 primers were HMPPARg2-F: 5′-atgggtgaaactctgggag-3′; and HMPPARg2-B: 5′-ccttgcatccttcacaagc-3′.

### Northern Blot Analysis

Total cytoplasmic RNA (10 μg) of 3T3-L1 cells harvested at days 0, 2, 4 and 8 of differentiation was glyoxylated and fractionated in 1.4% agarose gels in 10 mM Na2HPO4buffer (pH 7.0). After electrophoresis, the gel was blotted onto Hybond-N (Amersham), UV-cross-linked, and hybridised to 32P-labelled mouse SLUG and ap2 probes, respectively. Loading was monitored by reprobing the filter with a mouse 36B4 probe.

### Retroviral Infection

SLUG-deficient MEFs were infected with high-titers retrovirus stocks produced by transient transfection of φNX cells. The efficiency of infection was always >80%. The day before the infection, cells were plate at 2×10⁶ cells per 10-cm dish. Infected MEFs were selected for 3 d with 2.5 μg/mL of Puromycin (Sigma) and replated for the corresponding assay. The mouse SLUG cDNA was subcloned in the pQCXIP retrovirus (obtained from T. Jacks, Massachusetts Institute of Technology), as described (Bermejo-Rodriguez et al., 2006).

### Western Blot Analysis

Western blot analysis of different cells and tissues were carried out essentially as described (Castellanos et al., 1997). Extracts were normalized for protein content by Bradford analysis (Bio-Rad Laboratories, Inc., Melville, N.Y., USA) and Coommasie blue gel staining. Lysates were run on a 10% SDS-PAGE gel and transferred to a PVDF membrane. After blocking, the membrane was probed with the following primary antibodies: SLUG (G-18, Santa Cruz Biotechnology), PPARgamma (H-100 and E-8, Santa Cruz Biotechnology), RXRalpha (D-20, Santa Cruz Biotechnology), C/EBPbeta (C-19, Santa Cruz Biotechnology), C/EBPdelta (M-17, Santa Cruz Biotechnology), C/EBPalpha (14AA, Santa Cruz Biotechnology), and actin (1-19, Santa Cruz Biotechnology). Reactive bands were detected with an ECL system (Amersham).

### Luciferase Assays.

The reporter containing the proximal part of the hPPARγ2 promoter cloned in front of the luciferase gene (pGL3-hPPARg2p1000 vector) was kindly provided by Johan Auwerx (Fajas et al., 1997). The ratC/EBPαwtpSG5 and ratC/EBPβwtpSG5 expression vectors were kindly provided by Dr. Achim Leutz (Calkhoven et al., 2000). The expression vector pcDNA3-mSlug was generated by cloning the mouse Slug cDNA into the expression plasmid pcDNA3. For reporter assays, U2OS cells were transfected using Dual-Luciferase (Promega) with normalization to Renilla luciferase, and mean±standard error was determined from at least three data points. U2OS cells were maintained in Dulbecco's Modified Eagle Medium supplemented with 10% fetal bovine serum.

histone Acetylation Status.

Surgically-removed tissues from control, Combi-Slug and Slug-deficient mice were washed twice in ice-cold PBS supplemented with 5 mM Sodium Butyrate to retain levels of histone acetylation and homogenised in cold-TEB (PBS containing 0.5% Triton X-100 (v/v), 2 mM phenylmethylsulfonyl fluoride (PMSF) and 0.02% (v/v) NaN₃), place on ice 10 minutes with gentle stirring, centrifuge and wash in cold-TEB. Pellet was resuspended in 0.2N HCl and histones were extracted overnight at 4° C. Supernatant recovered by acidic extraction were subjected to SDS-polyacrylamide gel electrophoresis, transferred onto a polyvinylidene difluoride (PVDF) 22-μm pore size (Immobilon PSQ; Millipore) and immunoblotted with antiacetylated histone H3 (Upstate Biotechnology, Lake Placid, N.Y.). Core Histones loading control was performed with a classical Coumassie staining of acidic proteins extract. The signal was detected with enhanced chemiluminescence system (ECL; Amersham Pharmacia Biotech, UK limited) according to the protocols recommended by the manufacturer.

### Chromatin Immunoprecipitation (ChIP) Assay.

Mouse tissues (WAT and Liver) were surgical removed from different mice (wt, Combi-SLUG and SLUG−/−), homogenized and disaggregated in 2 mg/ml of Collagenase (Sigma, Type I) ON at 37° C. Cells were fixed in vivo at room temperature for 15 min by the addition of crosslinking mix (11% Formaldehyde; 100 mM NaCl; 0.5 mM EGTA; 50 mM HEPES, PH8.0) at a final concentration of 1% directly onto the tissue disaggregating media. Fixation was quenched by addition of glycine with a 0.125 M final concentration and the incubation was continued for a further 5 min. The cells were washed twice using ice-cold phosphate-buffered saline and collected. The cell pellets were washed and dissolved with cell lysis buffer (50 mM Tris-HCl (pH 8.0), 10 mM EDTA, pH 8.0; 1% SDS and a protease inhibitor cocktail (ROCHE)), and remained on ice for 10 min. The cell lysates were sonicated to shear chromosomal DNA with an average length between 500-1000 bp. After centrifugation to remove insoluble materials, the chromatin solution was diluted in a mixture of 9 parts dilution buffer (1% Triton X-100; 150 mM NaCl; 2 mM EDTA, pH8.0; 20 mM Tris-HCl, pH8.0 and a protease inhibitor cocktail (Sigma): 1 part lysis buffer, and the diluted solution was pre-cleared with protein G Sepharose beads on a rotating wheel at 4° C. for 1 h. Beads were removed by centrifugation and the supernatants were incubated with 2 mg of antibodies to HDAC (H-51), SLUG (H-140) or c/EBPα (14AA) from Santa Cruz Biotechnology Inc., (Santa Cruz, Calif., USA) or antiacetylated histone H3 at 4° C. overnight. The complexes were immunoprecipitated with protein G Sepharose beads 2 h at 4° C. The beads were washed once with IP dilution buffer, twice with wash buffer (20 mM Tris-HCl (pH 8.0), 150 mM NaCl, 2 mM EDTA, 1% Triton X-100, 0.1% SDS, and a protease inhibitor cocktail), once with final wash buffer (20 mM Tris-HCl (pH 8.0), 500 mM NaCl, 2 mM EDTA, 1% Triton X-100, 0.1% SDS, and a protease inhibitor cocktail), and twice with TE buffer. Immune complexes were eluted from the beads in the elution buffer (1% SDS; 100 mM NaHCO3) for 15 min. The proteins were removed from DNA by digesting with proteinase K and RNase A (500 μg/ml each) at 37° C. for 1 h. The crosslink was reversed by adding 5 M NaCl to a final concentration of 200 mM followed by incubation at 65° C. for 6 h. The sample DNAs were then extracted with phenol-chloroform-isoamyl alcohol (25:24:1), precipitated with cold-ethanol, and resuspended in TE buffer. Similarly purified DNA fragments from the chromatin extracts (input) were used as a control for PCR reactions. Precipitated DNAs were analysed by PCR of 30 cycles using primers: m-PPARγ2-ChIP-1F 5′-gtacagttcacgcccctcac-3′; m-PPARγ2-ChIP-1R 5′-tttgggagaggtgggaataa-3′; m-PPARγ2-ChIP-2F 5′-cagggaattattgccatctga-3′; and m-PPARγ2-ChIP-2R 5′-ggcaaggaattgtggtcagt-3′; m-PPARγ2-ChIP-3F 5′-cttgttgaataaatcacctt-3; m-PPARγ2-ChIP-3R 5′-cagtggcttttaaaatagaa-3′; covering 205, 212 and 219 bp, respectively, from PPARγ2 promoter. PCR products were separated on a 2% agarose gel and stained with ethidium bromide.

### Example 2

### Results

### SLUG is Expressed in White Fat in Humans

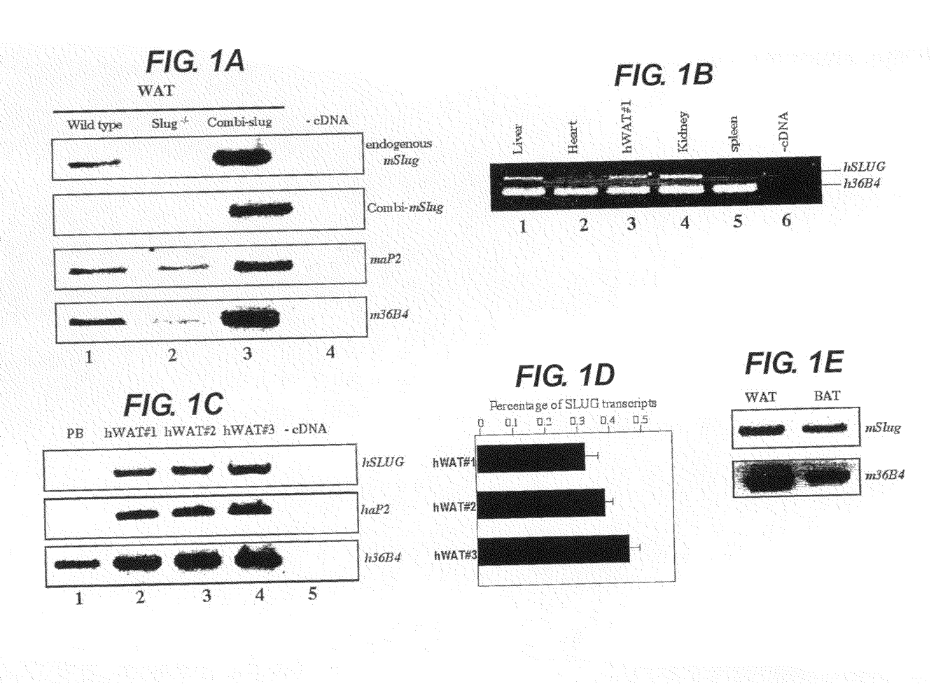

SLUG (SNAI2) expression and the effects of its deletion and overexpression are similar in mouse and human (Cohen et al., 1998; Perez-Losada et al., 2002; Sánchez-Martin et al., 2002; Oram et al., 2003; Sánchez-Martin et al., 2003; Perez-Mancera et al., 2005; Pérez-Mancera et al., 2006). Our previous observations indicated that SLUG was present in mouse adipose tissue (Perez-Mancera et al., 2005 and FIG. 1A-E). We now studied whether human adipose tissue expressed SLUG.

Expression of human SLUG was analyzed by reverse transcriptase (RT-PCR). The PCR products were transferred to a nylon membrane and analyzed by hybridization with a specific probe. SLUG expression was identified in human subcutaneous adipose tissues (FIGS. 1B and 1C). SLUG expression seems to be higher in donors with higher BMI (FIG. 1C, lane 2, BMI is normal; lane 3; BMI is considered overweight; and lane 4, BMI is considered obese) and this observation was confirmed by quantitative real time PCR (FIG. 1D). These observations indicate that expression of SLUG is a common finding in both human and mouse WAT, suggesting a role for SLUG in WAT development.

### SLUG Expression is Tightly Controlled During Adipocyte Differentiation

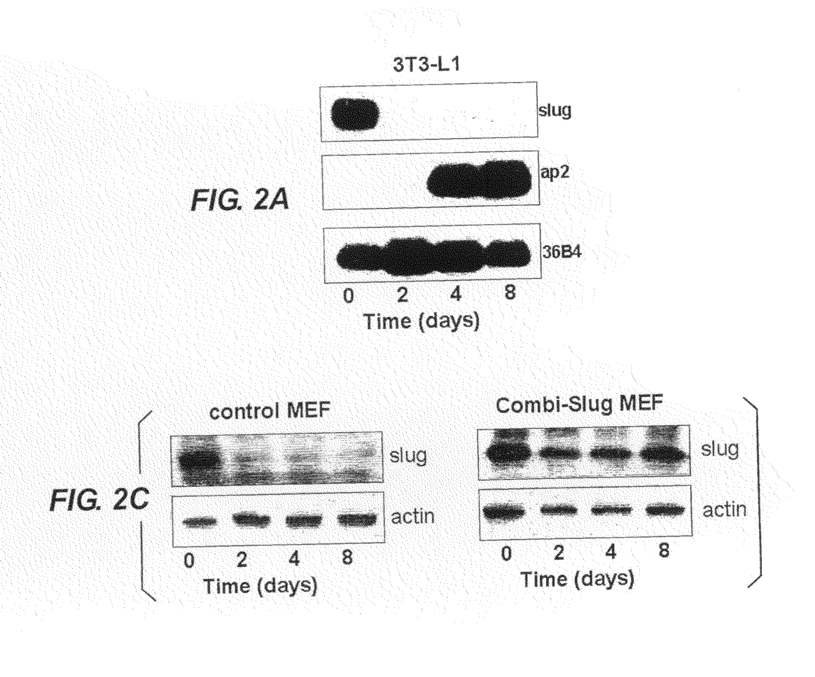

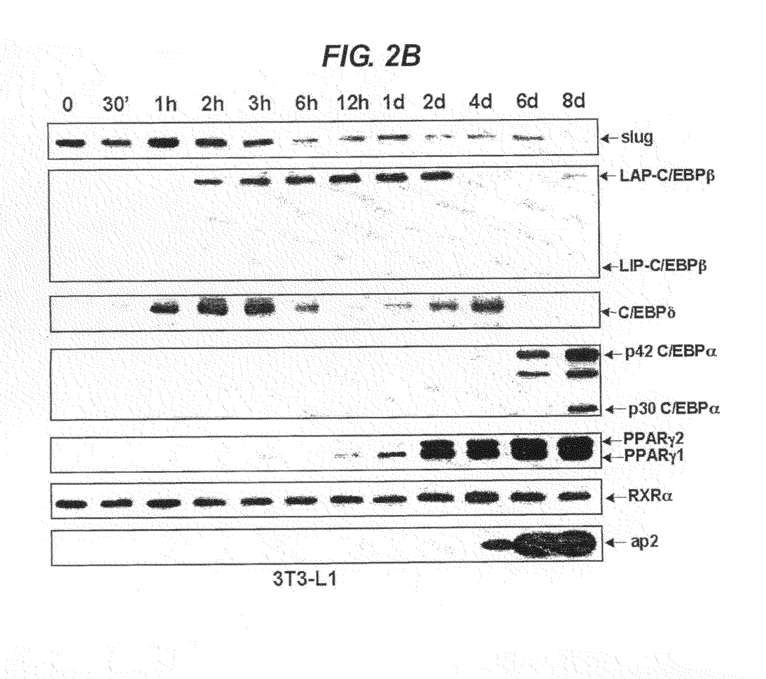

To determine the function of SLUG in WAT development, we first examined expression of SLUG during adipocyte differentiation. 3T3-L1 preadipocytes are a well-characterized in vitro model of adipocyte differentiation that can differentiate into mature adipocytes upon exposure to a mixture hormonal stimuli (Ntambi et al, 1988). SLUG expression is very high before differentiation treatment and the amount of SLUG mRNA and protein decreased during such hormonal stimulation (FIG. 2A-B), whereas the peroxisome proliferatoractivated factor γ (PPARγ), a transcription factor essential for adipocyte differentiation (Rosen et al., 1999), was apparent within 2 days and increased in abundance thereafter (FIG. 2B). This observation was further confirmed by using primary mouse embryonic fibroblasts as a model (FIG. 2C). These results indicate that SLUG is tightly controlled temporally and spatially during differentiation of preadipocytes.

### SLUG-Deficient Mice Exhibit Reduced WAT Mass

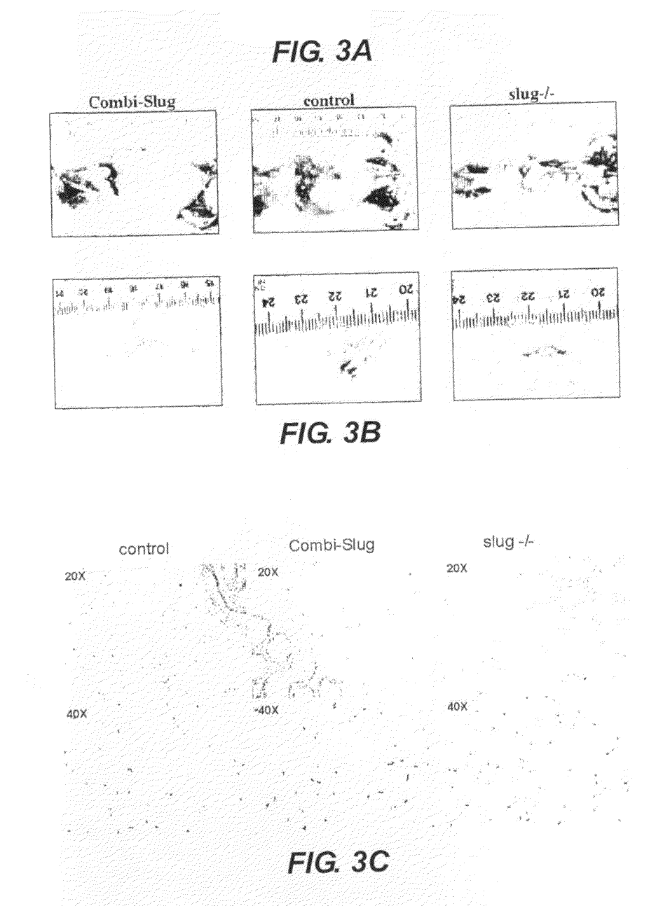

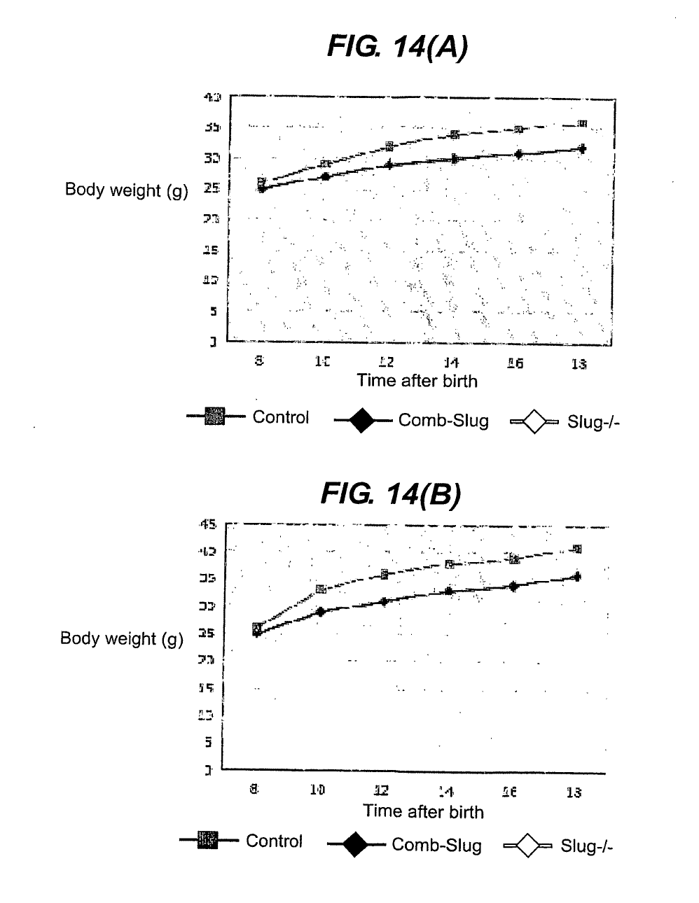

In order to determine the effect of SLUG expression in WAT development, we analyzed WAT mass in SLUG-deficient mite. We observed modest but significant reduction of body weight in Slug-deficient mice (FIG. 14). SLUG-deficient mice showed a large reduction in WAT weight in SLUG −/− mice (Table I and FIG. 3), but heterozygous mice were indistinguishable from wild-type mice. As control for decreased fat mass, we compared the muscle tissue of control and Slug −/− mice and we did not find differences. However, SLUG-deficient animals were protected against obesity induced by a high-fat diet (FIG. 14). In addition, food intake was similar in wild type (2.9±0.4 g per mouse per day) and SLUG-deficient mice (3.0±0.4 g per mouse per day). In the animals fed the high-fat diet, fat pads in SLUG-deficient mice showed no significant changes, but these pads had showed dramatic increases in the wild-type littermates, leading to an even more dramatic effect. This overall reduction in adipose tissue in SLUG-deficient mice was observed in males and females (Table 1). In contrast to WAT, other tissues including the interscapular brown adipose tissue (BAT), liver, and kidney had similar weights for wild-type and SLUG-deficient mice (Table 1).

TABLE 1
Adipose tissue mass in SLUG-deficient and SLUG-overexpressing mice

| | | Reproductive | Inguinal | Retroperitoneal |
|---|---|---|---|---|
| | Kidney | fat pad | fat pad | fat pad |
| Male | | | | |
| Wild type | 0.147 ± 0.009 | 0.78 ± 0.13 | 0.56 ± 0.10 | 0.30 ± 0.08 |
| Slug -/- | 0.149 ± 0.005 | 0.11 ± 0.07 | 0.11 ± 0.12 | 0.06 ± 0.03 |
| Combi- | 0.148 ± 0.008 | 0.175 ± 0.21 | 0.139 ± 0.13 | 0.77 ± 0.05 |
| SLUG | | | | |
| Female | | | | |
| Wild type | 0.143 ± 0.007 | ND | 0.55 ± 0.07 | 0.38 ± 0.09 |
| Slug -/- | 0.141 ± 0.009 | ND | 0.09 ± 0.10 | 0.08 ± 0.07 |
| Combi- | 0.144 ± 0.004 | ND | 0.144 ± 0.17 | 0.89 ± 0.11 |
| SLUG | | | | |

Mice were six months old. Weights are given in grams.
Values are mean ± SEM from five mice in each group.
Difference between “wild-type and Slug -/-” and “wild-type and Combi-SLUG” were statistically significant (P < 0.01) as determined by Mann-Whitney's test

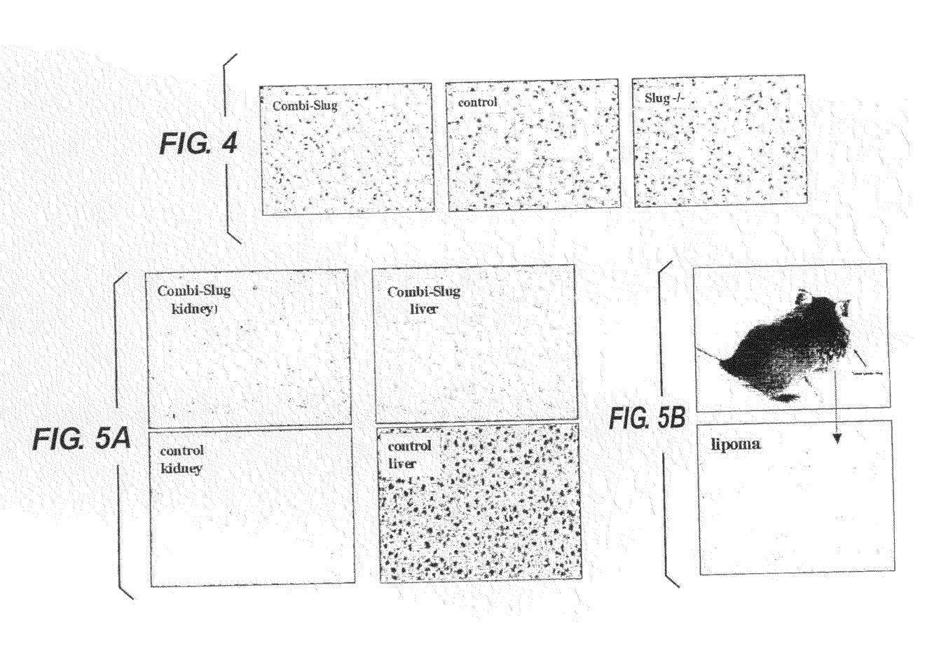

To characterise the phenotype of adipose tissue further, we examined histological sections of WAT and BAT (FIGS. 3 and 4). We observed no difference between the wild-type and KO mice in the BAT and WAT tissues. The histological analyses of the WAT in SLUG-deficient mice did not evidence any pathological change within the terminally differenciated adipocytes. On the contrary, SLUG-deficient mice had a normal architecture of the tissue and we did not observe any shift in the WAT toward immature in the SLUG-deficient mice.

To confirm that the decrease in WAT mass in SLUG-deficient mice was caused by the absence of SLUG, SLUG-deficient mice were crossed with Combi-SLUG mice (Perez-Mancera et al., 2005) that express the transgenic SLUG in WAT tissue (FIG. 1A). As expected, the WAT phenotype was rescued in the SLUG-deficient mice by expressing SLUG (Table 1).

We also investigated whether increased energy expenditure could account for the decrease in WAT mass in Slug-deficient mice by studying core body temperature and locomotor activity in these mice. We have measured locomotor activity and body temperature in Slug-deficient mice (Table 2), showing no significant differences.

TABLE 2
Locomotor activity and body temperature in Slug-deficient and Slug-
overexpressing mice.

| | % activity time^{a} | | |
|---|---|---|---|
| | Lights-on | Lights-off | Rectal temperature^{b}, ° C. |
| Male | | | |
| Wild type | 42.2 ± 4.5 | 59.5 ± 2.3 | 37.58 ± 0.04 |
| Slug -/- | 41.7 ± 3.5 | 60.4 ± 3.9 | 37.56 ± 0.05 |
| Combi-SLUG | 43.1 ± 5.6 | 62.3 ± 4.7 | 37.60 ± 0.03 |
| Female | | | |
| Wild type | 43.6 ± 3.7 | 64.5 ± 4.4 | 37.71 ± 0.03 |
| Slug -/- | 42.3 ± 6.1 | 65.6 ± 5.9 | 37.73 ± 0.04 |
| Combi-SLUG | 43.9 ± 4.8 | 66.1 ± 5.3 | 37.72 ± 0.04 |

Mice were four months old.
Values are means ± SEM from six mice in each group.
Differen between “wild-type and Slug -/-” and “wild-type and Combi-Slug” were not statistically significant as determined by Mann-Whitney's test.
^{a}Time spent in activity during the lights-on and lights-off periods in wilt-type, Slug -/- and Combi-Slug mice. Activity was defined as displacement of at least 1 cm (n = 5). The method of measuring locomotor activity has been described (Aminian et al., 1993). In brief, two co-ordinates of the animal's centre of mass were determined by an opto-electronic device consisting of an infared light emitting diode and receiver. The home- cage travelled distance was measured in male and female mice 10 to 12 weeks of age, for 500 minutes during either the lights-off or -on period. Quantitative analysis of the fraction of time spent in activity was done by measuring the time during which the animal showed a displacement of at least 1 cm.
^{b}Metod of measuring body temperature: rectal temperature were taken using a lubricated clinical thermometer inserted to a depth of ~1 cm and left in place until a stable reading was obtained (apoprox. 1 minute)

### SLUG Expression Modulates White Adipose Tissue Size in Mice

The above results suggest that SLUG controls WAT tissue size. Thus, we next evaluated the effect of upregulation of SLUG expression in WAT mass in vivo. Mice carrying a tetracycline-repressible SLUG transgene (Combi-SLUG mice) were initially generated to investigate the potential role of SLUG overexpression in cancer (Perez-Mancera et al., 2005). As anticipated from the patterns of SLUG expression, Combi-SLUG mice expressed high amounts of SLUG in adipose tissue (FIG. 1A). We now analyzed WAT in SLUG-overexpressing mice. Transgenic mice kept off doxycycline from conception, leading to SLUG expression throughout development, were found to have a modest but significant increased in body weight (FIG. 14) and to have strikingly increased WAT mass. Uniformly, male and female SLUG-overexpressing mice show a significant increase in WAT weight (Table 1 and FIG. 3), indicating that the overexpression of SLUG does perturb normal WAT development. Moreover, food intake in Combi-SLUG mice (2.9±0.6 g per mouse per day) was similar to wild type mice.

We also investigated whether decreased energy expenditure could account for the increase in WAT mass in Slug-Combi mice by studying core body temperature and locomotor activity in these mice. We have measured locomotor activity and body temperature in Combi-Slug mice (Table 2), showing no significant differences.

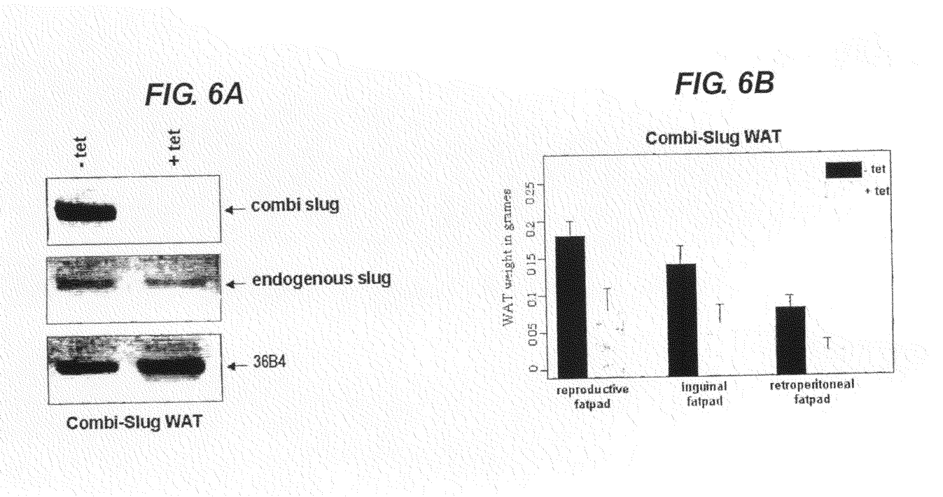

Some Combi-SLUG animals presented (12%) lipid accumulation in kidney and liver (FIG. 5A) and developed palpable masses involving the adipose tissues, which, upon dissection and histological examination revealed lipoma formation (FIG. 5B). Similarly to SLUG-deficient mice, the histological analyses of the white adipose depots in the SLUG-overexpressing animals revealed a normal architecture of the tissue, (FIG. 3). However, the volumes of adipocytes of Combi-Slug mice were larger than those of normal mice (FIG. 3C). Pathological changes were not observed in the brown adipose tissue (BAT) of these transgenic mice (FIG. 4). Thus, SLUG overexpressing mice exhibit increased WAT mass. This result is in agreement with the observation that SLUG expression seems to increase in parallel with BMI in humans (FIGS. 1C and 1D). In fact, the physiological modulation of Combi-SLUG expression during the course of adipocyte differentiation is lost in Combi-SLUG cells (FIG. 2C).

The above results support the hypothesis that SLUG expression modulates adipose tissue size. Therefore abolition of SLUG overexpression might be expected to either halt or reduce WAT increase. To assess this, twelve Combi-SLUG mice with an increase in body weight compared to wild-type mice were evaluated for WAT size following administration of tetracycline (4 gr/L in the drinking water for 3 weeks, a dose sufficient to suppress of exogenous SLUG expression—FIG. 6A—). Eleven out of 12 CombitTA-SLUG mice exhibited a decrease in body weight, being the WAT weight similar to wild-type mice (FIG. 6B). Thus, these results indicate that the WAT alterations, induced by SLUG, are reversible.

### Expression of Adipogenic Genes in WAT of SLUG-Deficient and Combi-SLUG Mice.

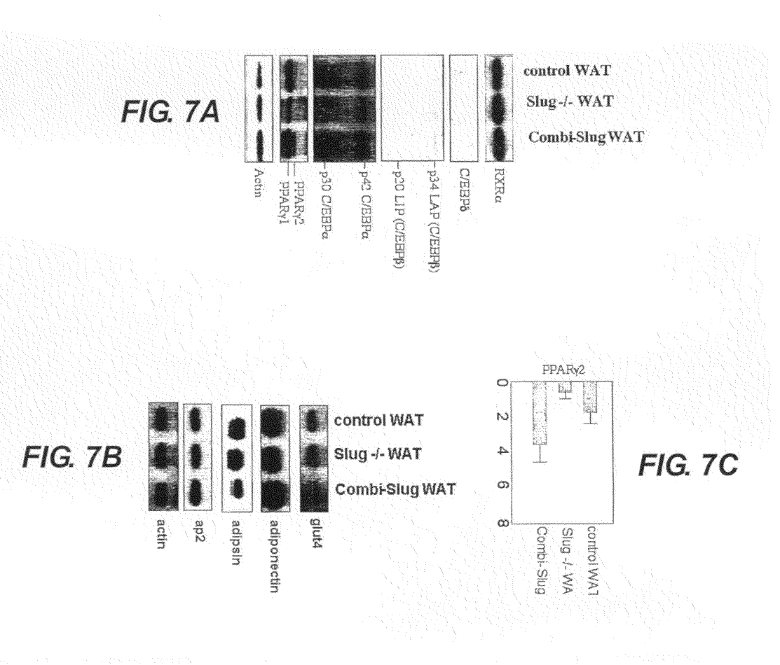

The development of adipose tissue involves a differentiation switch that activates a new program of gene expression, followed by accumulation of lipids in a hormone-sensitive manner (Morrison and Farmer, 2000; Rosen et al., 2000). To further explore the molecular basis through which SLUG favours and lack of SLUG impairs the development of fat tissue, we examined the expression levels of the proteins responsible for WAT development (FIG. 7A-B) and the expression levels of several adipocyte markers (FIG. 7C). As shown in FIG. 7, the expression of RXR [symbol] , C/EBP™, C/EBP® and C/EBP [symbol] seem not to be affected. However, the expression of PPARγ2 was decreased in the WAT of SLUG-deficient mice and increased in the WAT of Combi-SLUG mice (FIG. 7B). Taken together, these results suggest that SLUG could modulate WAT development by affecting PPARγ2 expression.

### Impaired In Vitro Adipogenesis of SLUG-Deficient and Combi-SLUG MEFs: SLUG Regulates Adipocyte Differentiation via PPARγ2.

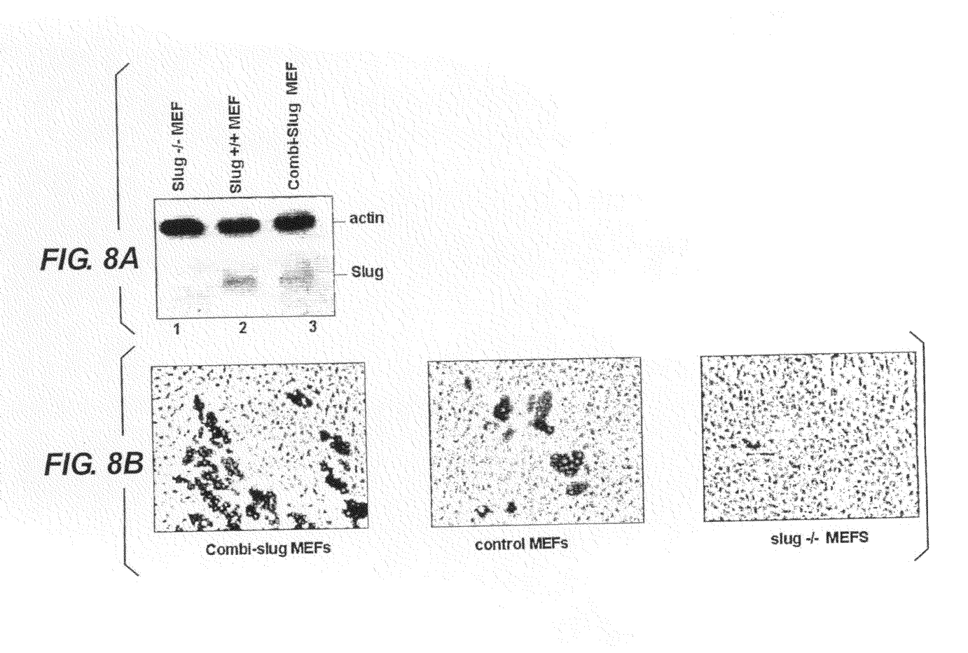

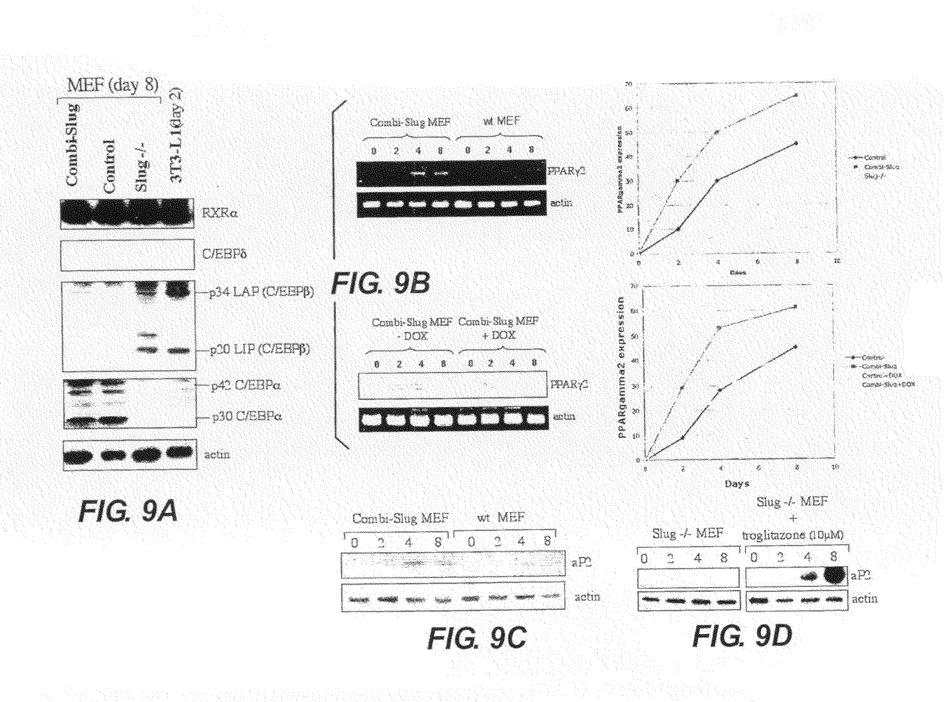

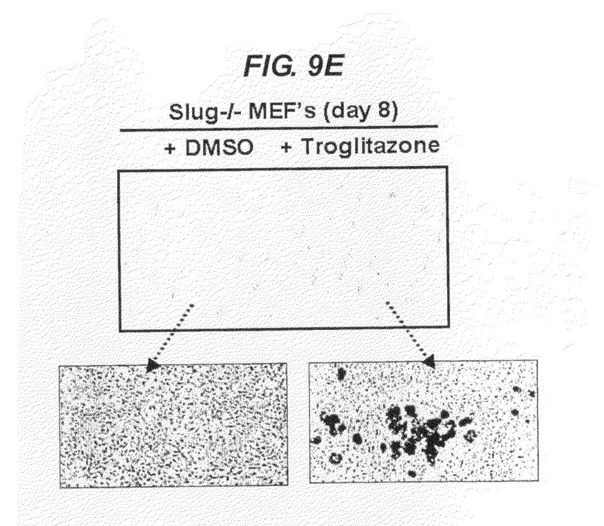

The adipogenesis of MEFs by hormonal induction is a well-established model system for the study of adipocyte differentiation in vitro (Tontonoz P et al., 1994; Wu et al., 1999). To further examine the contribution of SLUG to adipogenesis, we isolated MEFs from days 13.5 of SLUG −/−, Combi-SLUG and control embryos (FIG. 8A). At day 8 after hormonal induction, there is lipid accumulation, defined as percentage of cells that are oil-red-O positive in control MEFs (15-25%). However, there was extensive accumulation in Combi-SLUG MEFs (35-45%), and barely any lipid accumulation in SLUG-deficient MEFs (0.1-0.5%) (FIG. 8B). In agreement with these morphological changes, the marker of adipogenesis, PPARγ2, was also significantly reduced in the hormone-induced SLUG −/− MEFs (FIG. 9A-B) and increased in Combi-SLUG MEFS (FIG. 9A-B), compared to those in SLUG +/+ MEFs. This increase in PPARγ2 expression was reverted upon doxycycline treatment of Combi-Slug MEFs (FIG. 9B). Similarly, the expression of the fat cell maker, apt, confirmed the morphological changes (FIGS. 9C-D). To define whether the stimulation of PPARγ can rescue adipogenesis in SLUG-deficient cells, adipocytic differentiation was induced in SLUG −/− adipocyte differentiation block in SLUG-deficient MEFs was normalised by treatment with the PPARγ agonist troglitazone (FIG. 9D-E).

These results indicate that SLUG modulates adipogenesis in vitro by affecting PPARγ2 expression.

### The Adipogenesis Defects in SLUG −/− MEFs can be Rescued by Ectopic Expression of SLUG.

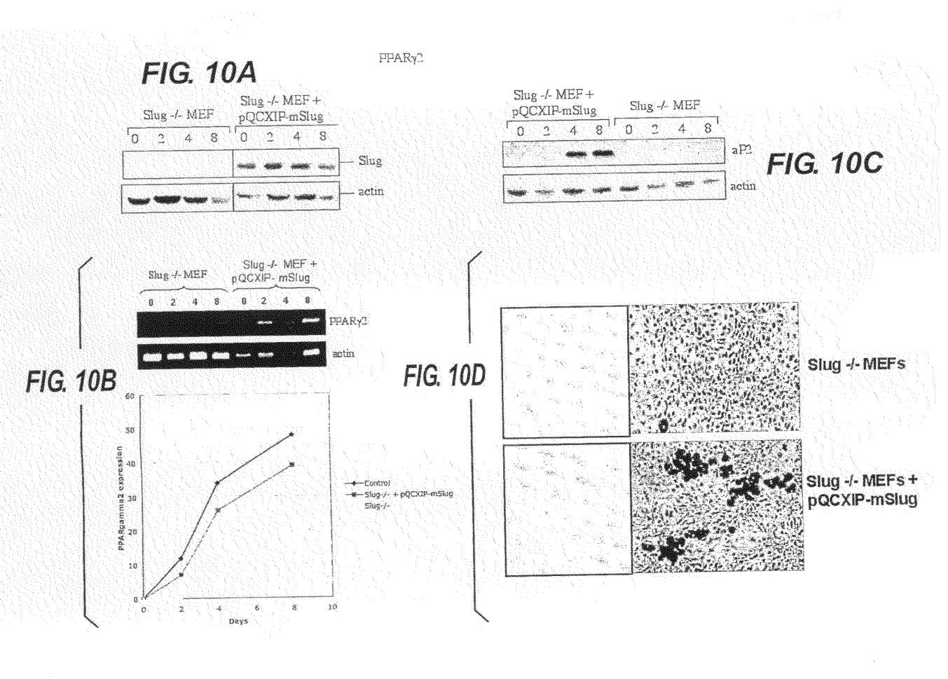

Our data revealed that PPARγ2 expression is modulated by SLUG and these data were confirmed by normalization of the adipocyte differentiation capacity of SLUG-deficient cells by troglitazone, suggesting an interesting link between this gene and SLUG. In order to confirm this transcriptional regulation we re-introduced wild-type SLUG in SLUG-deficient MEFs by retroviral transduction and evaluated the expression level of PPARγ2 by Western analyses. Retrovirus-mediated expression of SLUG in SLUG-deficient MEFS re-established the aberrant expression of PPARγ2 and adipocyte differentiation capacity to wild-type levels as shown in FIG. 10A-B. The demonstration that SLUG was sufficient to fully recover its aberrant expression in cells lacking SLUG further indicates that PPARγ2 was regulated directly by SLUG.

Slug does not Transactivate the PPARγ2 Promoter.

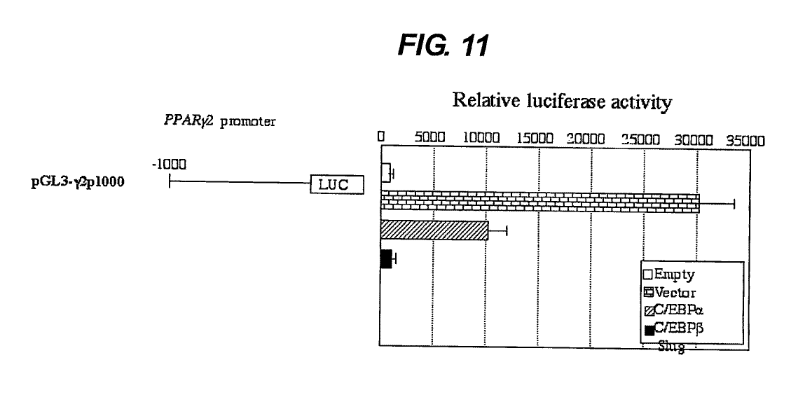

Because the results so far suggest that Slug directly regulates PPARγ2 expression, we examined whether Slug might be directly involved in the control of PPARγ2 transcription. A 1 kb proximal promoter region of human PPARγ2 was previously shown to be sufficient to drive the PPARγ2′ s expression in reporter assays (Fajas et al., 1997) and it is active in U2OS cells when co-transfected with C/EBPα and C/EBPβ expression vectors (FIG. 11). To directly assess the ability of Slug to activate transcription from DNA sequences present in the PPARγ2 promoter, an expression vector containing a Slug cDNA was co-transfected into U2OS cells along with the reporter vector containing the PPARγ2 promoter (pGL3-hPPARf2p1000 vector). Co-expression of Slug did not increase luciferase activity compared to the activity with the empty vector (FIG. 11).

### Histone Modifications in WAT of Combi-Slug and Slug-Deficient Mice.

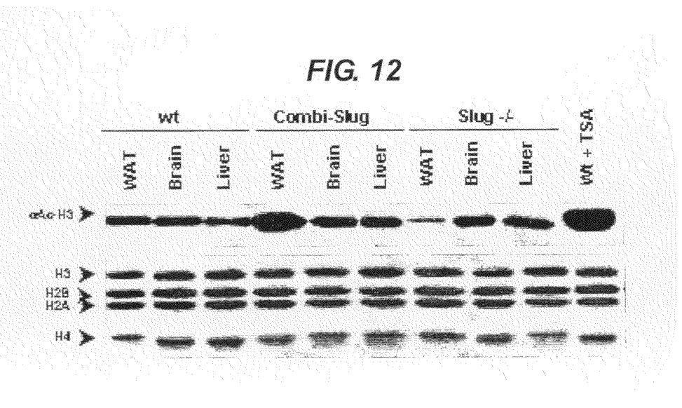

The above results suggest that Slug does not have a direct role in inducing expression of PPARγ2 through association with regulatory elements in the PPARγ2 gene promoter. However, programmed regulation of gene expression is the result of coordinated modulation of the transcription machinery and chromatin-remodeling factors, notably histone acetylation and deacetylation. To address this issue, we measured the histone acetylation status in WAT of Combi-Slug and Slug-deficient mice. We analyzed the acetylation levels at histone H3 using protein blotting. We found a high increase in histone H3 acetylation in Combi-Slug WAT and a decrease in histone H3 acetylation in Slug −/− WAT compared with control mice (FIG. 12). Thus, these results show a correlation between SLUG expression and histone acetylation status in adipose tissue.

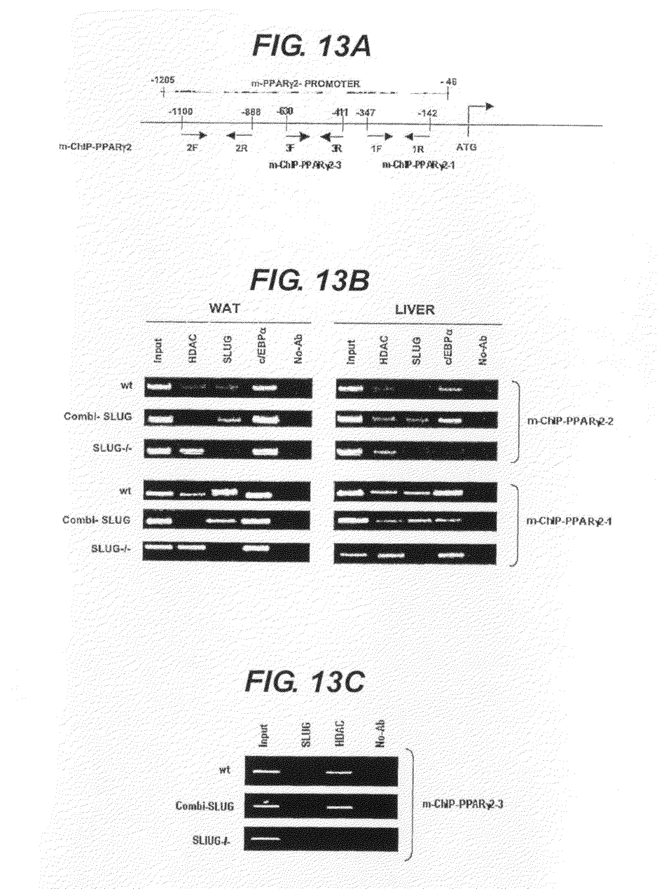

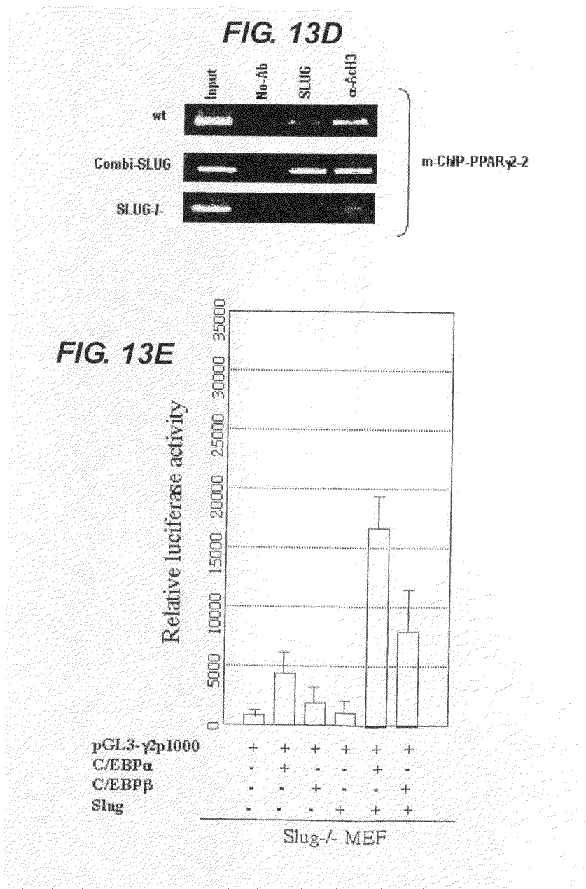

Recent work has implicated histone deacetylases (HDAC) as mediators of the gene regulation modulated by Slug (Peinado et al., 2004; Bermejo-Rodríguez et al., 2006). The major function of HDAC is to remove acetyl groups from histones, which results in condensation of the chromatin structure (Ayer, 1999). This, in turn, diminishes the access of transcription factors to the target DNA and ultimately leads to transcriptional repression. To explore whether Slug is indeed recruited at the PPARγ2 gene promoter inside the cell nucleus, we performed a chromatin immunoprecipitation (ChIP) assay (FIG. 13A). Chromatin samples were prepared from WAT of control, Combi-Slug and Slug-deficient mice, and then immunoprecipitated with specific antibodies against C/EBPα, Slug and HDAC1. The binding of C/EBPα was detectable, which is consistent with the current model of PPARγ2 control by C/EBPs (FIG. 13B). This ChIP analysis revealed that not only Slug but also HDAC1 is recruited at the PPARγ2 promoter in control adipose tissue (FIG. 13B). Importantly, HDAC1 is not recruited in the nucleus in WAT cells from Combi-Slug mice, in agreement with the abundance of acetylated histones at WAT of Combi-Slug mice (FIG. 12). On the other hand, HDAC1 is recruited at the PPARγ2 promoter in Slug-deficient WAT (FIG. 13B). Thus, these data show a differential HDAC recruitment to the PPARγ2 promoter in a tissue- and Slug-dependent manner. These findings predict that increase Slug expression may also lead to increase acetylation at the PPARγ2 promoter and viceversa. To test this, we determined histone H3 acetylation at the PPARγ2 promoter in WAT of control, Combi-Slug and Slug-deficient mice (FIG. 13D). The ChIP analysis showed a differential H3 acetylation at the PPARγ2 promoter in a Slug-dependent manner, suggesting a change in the PPARγ2 chromatin toward a more active and “open” state in the Combi-Slug WAT and toward an inactive state in the Slug-deficient WAT. If this were the case then C/EBPs might be expected to be less able to transactivate the PPARγ2 reporter in the absence of Slug. To directly assess this, an expression vector containing either a C/EBPα or a C/EBPβ cDNA was co-transfected into Slug-deficient cells along with the reporter vector containing the PPARγ2 promoter (pGL3-hPPARg2p1000 vector). Co-expression of C/EBPα or C/EBPβ increased luciferase activity compared to the activity with the empty vector although they were not very efficient (FIG. 13E). However, co-expression of Slug was able to increase the luciferase activity induced by C/EBPα or C/EBPβ (FIG. 13E). These observations may explain the differences in, PPARγ2 expression in Slug-deficient and Combi-Slug mice and the role of Slug in WAT development.

### Example 3

### Discussion

In mammals, cell specification is a process in which cells first become committed to a developmental fate, after which they differentiate and acquire the properties of a specific cell type. Adipocyte development is controlled by a genetic programme that leads fibroblasts to become preadipocytes. When further induced, preadipocytes differentiate and express genes that allow them to store lipid and become mature adipocytes. While many of the components of the gene regulatory network that controls differentiation of adipocytes have been elucidated in studies of cultures 3T3-L1, little is known about the developmental signals that control the development of adipocytes in vivo. The present study establishes for the first time the important role that is played by SLUG in adipogenesis in vivo and in vitro.

SLUG expression is tightly controlled during adipocyte differentiation. SLUG is expressed in vivo but is only expressed transiently in culture cells, suggesting that it may play a role in initiating and/or maintaining adipogenesis in vivo. Expression of SLUG was observed before the induction of differentiation in 3T3-L1 cells (which are lineage-determined preadipocytes) and MEFs (which are uncommitted progenitor cells) and found to be downregulated within two days after applying the hormonal stimuli in both cell types. A similar expression pattern is observed in the hematopoietic system where uncommitted progenitor cells differentiate to mature cells, at which time the expression of SLUG is downregulated (Inoue et al., 2002; Perez-Losada et al, 2002).

These findings indicate that SLUG downregulation is required to initiate adipogenesis, suggesting it could play a role in the development or maintenance of these cells from precursor cells. The reduced WAT mass observed in SLUG-deficient mice and the reduced adipocyte differentiation seen in SLUG-deficient MEFs are also consistent with a role for SLUG in early adipocyte differentiation, although the present experiments cannot distinguish function of SLUG in preadipocytes from an effect on lineage commitment. The dissection of the mechanisms controlling its expression could lead in turn to the identification of signals that control adipogenesis in vivo. Of note, Kit is one of the markers for presumptive mesenchymal stem cells as well as being an activator of SLUG expression (Perez-Losada et al, 2002). Moreover, several SLUG targets have been implicating in regulating stem cell finction (Bermejo-Rodriguez, 2006).

The regulation of these genes by SLUG could be important in maintaining uncommitted progenitor cells. It appears that SLUG must be kept above a certain threshold level to achieve normal WAT development both in vivo and in vitro. Consistent with this interpretation, mice carrying a tetracycline-repressible SLUG transgene (Combi-SLUG) exhibit an increase in WAT size, that was specifically re-established by suppression of the SLUG transgene. Consistent with in vivo data, Combi-SLUG MEFs increased adipocyte differentiation, suggesting that this factor positively regulates adipocyte differentiation. Thus, it seems likely that failure to regulate SLUG expression explains why Combi-SLUG mice develop obesity.

The data presented here indicate that SLUG is a novel mediator of adipose tissue development in mammals. We therefore analyzed the molecular mechanism by which SLUG controls WAT development. It is well defined that C/EBPβ can promote the fat differentiation of culture cells. After exposure to a hormonal cocktail, CEBPβ is actively expressed and then begins to diminish around day 2 of hormonal induction, at which point the expression of C/EBPα and PPARγ increase (Cao et al, 1991). C/EBPα and PPARγ induce programs of gene expression leading to the differentiation of mature adipocytes (Lin and Lane, 1994; Tontonoz et al., 1994; Rosen et al., 2002). It has been documented that selective disruption of PPARγ2 impairs the development of adipose tissue and is absolutely required for differentiation (Zhang et al., 2004), while C/EBPα® is not strictly required for adipogenesis (Rosen et al., 2002). In vivo and in vitro Combi-SLUG and SLUG-deficient tissues and MEFs exhibit normal expression of C/EBP factors. However, we found that PPARγ2 expression is altered both in vivo in WAT of SLUG-deficient and Combi-SLUG mice and in vitro in SLUG-deficient MEFs and Combi-SLUG MEFs during the course of adipocytic differentiation. Complementation studies in SLUG-deficient MEFs confirmed this regulation, although Slug was not able to activate transcription from a reporter vector containing the PPARγ2 promoter. However, when we measured the histone acetylation status in WAT of Combi-Slug and Slug-deficient mice, we identified a correlation between Slug gene expression and histone acetylation status in adipose tissue. This observation close to recent work implicating HDAC as mediators of the gene regulation modulated by Slug (Peinado et al., 2004; Bermejo-Rodríguez et al., 2006) prompted us to explore whether Slug is indeed recruited at the PPARγ2 gene promoter. Our ChIP experiments showed that Slug and HDAC1 are bound to the endogenous PPARγ2 promoter in intact chromatin in WAT, and identified a differential HDAC recruitment to the PPARγ2 promoter in a tissue- and Slug-dependent manner. In agreement with these observations, the ChIP analysis confirmed a differential H3 acetylation at the PPARγ2 promoter in a Slug-dependent manner. Thus, the most straightforward model for the Slug requirement for PPARγ2 gene expression would be that a lack of Slug binding to the PPARγ2 gene results in the formation of a silencing complex that represses the expression of the gene by histone deacetylation. On the contrary, HDAC1 is not recruited at the PPARγ2 promoter in WAT cells from Combi-Slug mice, in agreement with the abundance of acetylated H3 histones at WAT of Combi-Slug mice (FIGS. 12, and 13D). This, in turn, will increase the access of transcription factors to the target DNA and ultimately leads to PPARγ2 transcriptional activation (FIG. 13E). This could be a potentially relevant clinical issue, as HDAC inhibitors are drugs that have activity at doses that are well tolerated by patients in clinical trials (Marks and Jiang, 2005). In agreement with this model, it has been shown that down-regulation of histone deacetylases stimulates adipocyte differentiation (Yoo et al., 2006).

The expression of SLUG in human WAT tissue (which seems to be higher in individuals with higher BMI-mimicking Combi-SLUG mice) is of relevance in human obesity, particularly when the obesity observed in Combi-Slug mice is associated with adipose cell hypertrophy. The WAT size in Combi-Slug mice can be reverted by suppressing Slug expression and the WAT size is reduced in Slud-deficient mice. SLUG is overexpressed in other human diseases like cancer (Inukai et al., 1999; Khan et al., 1999; Perez-Mancera et al., 2005). Although white fat is a non-malignant tissue, it has the capability to proliferate quickly and expand (Wasserman, 1965; Cinti, 2000).

Thus, SLUG expression might therefore define a common pathway for cancer and obesity. However, the role conferred by SLUG is reversible in obesity. SLUG has been shown to play similar roles to Snail in several systems, and, thus, other members of the Snail family of transcription factors could also been involved in similar biological functions to those described herein to SLUG. But is not clear whether this functional equivalence also occurs during adipogenesis. Among previously identified SLUG-regulated species, the related transcription factor Snail was reported as SLUG induced in Xenopus (Aybar et al., 2003). However, SLUG does not influence the expression of Snail in MDCK cells (Bolos et al., 2003) and in MEFs (Bermejo-Rodriguez et al, 2006). Similarly, we did not detect a change in Snail expression associated to overexpression or deficiency of SLUG in mice.

In summary, we report the identification of SLUG as playing an essential role in adipose tissue development and differentiation. An analysis of its regulation in vivo could lead to a fuller understanding of regulation of adipogenesis. Our results connect adipogenesis with the requirement of a critical level of an EMT regulator in mammals. Because SLUG modulates adipose tissue size in mice and is also expressed in human white fat, these results will help to develop a strategy that would form the basis for improved antiobesity and antilipodystrophy therapies.

### REFERENCES

Aybar M J, Nieto M A and Mayor R. (2003). Snail precedes SLUG in the genetic cascade required for the specification and migration of the Xenopus neural crest. Development 130, 483-494.
Ayer D E. (1999) Histone deacetylases: transcriptional repression with SINers and NuRDs. Trends Cell Biol. 19(5):193-198.
Bolos V, Peinado H, Perez-Moreno M A, Fraga M F, Esteller M, and Cano A. (2003). The transcription factor SLUG represses E-cadherin expression and induces epithelial to mesenchymal transitions: a comparison with Snail and E47 repressors. J Cell Sci. 116, 499-511.
Bermejo-Rodríguez, C., Pérez-Caro, M., Pérez-Mancera, P. A., Sánchez-Beato, M., Piris, M. A., and Sánchez-García, I. (2006) Mouse cDNA Microarray Analysis Uncovers SLUG Targets in Mouse Embryonic Fibroblasts. Genomics 87, 113-118.
Calkhoven C F, Muller C, Leutz A. (2000) Translational control of C/EBPalpha and C/EBPbeta isoform expression. Genes Dev. 14 (15), 1920-1932.
Cao Z, Umek R M, and McKnight S L. (1991). Regulated expression of three C/EBP isoforms during adipose conversion of 3T3-L1 cells. Genes Dev. 5(9), 1538-1552.
Castellanos, A., Pintado, B., Wuruaga, E., Arévalo, R., López, A., Orfao, A., and Sánchez-García, I. (1997). A BCR-ABL(p190) fusion gene made by homologous recombination causes B-cell acute lymphoblastic leukemias in chimeric mice with independence of the endogenous bcr product. Blood 90, 2168-2174.
Chen Z, Torrens J I, Anand A, Spiegelman B M, and Friedman J M. (2005). Krox stimulates adipogenesis via C/EBPbeta-dependent and -independent mechanisms. Cell Metab. 1(2), 93-106.
Cinti, S. (2000). Anatomy of the adipose organ. Eat. Weight Disord. 5, 132-142.
Cohen M. E., Yin M., Paznekas W. A., Schertzer M., Wood S., and Jabs E. W. (1998). Human SLUG gene organization, expression, and chromosome map location on 8q. Genomics 51(3), 468-471.
Fajas L, Auboeuf D, Raspe E, Schoonjans K, Lefebvre A M, Saladin R, Najib J, Laville M, Fruchart J C, Deeb S, Vidal-Puig A, Flier J, Briggs M R, Staels B, Vidal H, Auwerx J. (1997) The organization, promoter analysis, and expression of the human PPARgamma gene. J Biol. Chem. 272(30), 18779-89.
Inukai T., Inoue A., Kurosawa H., Goi K., Shinjyo T., Ozawa K., Mao M., Inaba T., and Look A. T. (1999). SLUG, a ces-1-related zinc finger transcription factor gene with antiapoptotic activity, is a downstream target of the E2A-HLF oncoprotein. Mol. Cell. 4(3), 343-352.
Inoue A., Seidel M. G., Wu W., Kamizono S., Ferrando A. A., Bronson R. T., Iwasaki H., Akashi K., Morimoto A., Hitzler J. K., Pestina T. I., Jackson C. W., Tanaka R., Chong M. J., McKinnon P. J., Inukai T., Grosveld G. C., and Look A. T. (2002). SLUG, a highly conserved zinc finger transcriptional repressor, protects hematopoietic progenitor cells from radiation-induced apoptosis in vivo. Cancer Cell 2(4), 279-288.
Jiang R., Lan Y., Norton C. R., Sundberg J. P., and Gridley T. (1998) The SLUG gene is not essential for mesoderm or neural crest development in mice. Developmental Biology 198, 277-285.
Khan, J., Bittner, M. L., Saal, L. H., Teichmenn, V., Azorsa, D. O., Gooden, G. C., Pavan, W. J., Trent, J. M., and Meltzer, P. S. (1999). cDNA microarrays detect activation of a myogenic transcription program by the PAX3-FKHR fusion gene. Proc. Natl. Acad. Sci. USA 96, 13264-13269.
Lin F T, and Lane M D. (1994). CCAAT/enhancer binding protein alpha is sufficient to initiate the 3T3-L1 adipocyte differentiation program. Proc Natl Acad Sci USA. 91(19), 8757-8761.
Marks P A, Jiang X. (2005) Histone deacetylase inhibitors in programmed cell death and cancer therapy. Cell Cycle 4(4): 549-551.
Morrison R F, and Farmer S R. (2000). Hormonal signaling and transcriptional control of adipocyte differentiation. J. Nutr. 130(12), 3116S-3121S.
Ntambi J M, Buhrow S A, Kaestner K H, Christy R J, Sibley E, Kelly T J Jr, and Lane M D. (1988). Differentiation-induced gene expression in 3T3-L1 preadipocytes. Characterization of a differentially expressed gene encoding stearoyl-CoA desaturase. Biol. Chem. 263(33), 17291-17300.
Oram K F, Carver E A, and Gridley T. (2003). SLUG expression during organogenesis in mice. Anat Rec. 271A(1), 189-191.
Peinado H, Ballestar E, Esteller M, Cano A. (2004) Snail mediates E-cadherin repression by the recruitment of the Sin3A/histone deacetylase 1 (HDAC1)/HDAC2 complex. Mol Cell Biol. 24(1), 306-319.
Perez-Losada J., Sanchez-Martin M., Rodriguez-Garcia A., Sanchez M. L., Orfao A., Flores T., and Sanchez-Garcia I. (2002). Zinc-finger transcription factor SLUG contributes to the function of the stem cell factor c-kit signaling pathway. Blood 100(4), 1274-1286.
Perez-Losada J., Sanchez-Martin M., Perez-Cam M., Perez-Mancera P. A., and Sanchez-Garcia I. (2003). The radioresistance biological function of the SCF/kit signaling pathway is mediated by the zinc-finger transcription factor SLUG. Oncogene 22(27), 4205-4211.
Pérez-Mancera P A, González-Herrero I, Pérez-Caro M, Gutierrez-Clanca N, Flores T, Gutierrez-Adan A, Pintado B, Sanchez-Martin M, and Sanchez-Garcia I. (2005). SLUG (SNAI2) in cancer development. Oncogene 24 (19), 3073-3082.
Perez-Mancera P A, Gonzalez-Herrero I, Maclean K, Turner A M, Yip M Y, Sanchez-Martin M, Garcia J L, Robledo C, Flores T, Gutierrez-Adan A, Pintado B, and Sanchez-Garcia I. (2006). SLUG (SNAI2) overexpression in embryonic development. Cytogenet Genome Res. 114(1), 24-29.
Rosen E D, Sarraf P, Troy A E, Bradwin G, Moore K, Milstone D S, Spiegelman B M, and Mortensen R M. (1999). PPAR gamma is required for the differentiation of adipose tissue in vivo and in vitro. Mol. Cell. 4(4), 611-617.
Rosen E D, Walkey C J, Puigserver P, and Spiegelman B M. (2000). Transcriptional regulation of adipogenesis. Genes Dev. 14(11), 1293-307.
Rosen E D, Hsu C H, Wang X, Sakai S, Freeman M W, Gonzalez F J, Spiegelman B M. (2002). C/EBPalpha induces adipogenesis through PPARgamma: a unified pathway. Genes Dev. 16(1), 22-26.
Sanchez-Martin M., Rodriguez-Garcia A., Perez-Losada J., Sagrera A., Read A. P., and Sanchez-Garcia I. (2002). SLUG (SNAI2) deletions in patients with Waardenburg disease. Hum Mol. Genet. 11(25), 3231-3236.
Sanchez-Martin M., Perez-Losada J., Rodriguez-Garcia A., Gonzalez-Sanchez B., Korf B. R., Kuster W., Moss C., Spritz R. A., and Sanchez-Garcia I. (2003). Deletion of the SLUG (SNAI2) gene results in human piebaldism. Am J Med. Genet. 122A(2), 125-132.
Sanchez-Martin M., Gonzalez-Herrero I., and Sanchez-Garcia, I. SNAI2 (SNAIL HOMOLOG 2). Atlas Genet Cytogenet Oncol Haematol. April 2004, (http://www.infobiogen.fr/services/chromcancer/Genes/SNAUID453.html).
Sefton M., Sanchez S., and Nieto M. A. (1998). Conserved and divergent roles for members of the Snail family of transcription factors in the chick and mouse embryo. Development 125(16), 3111-3121.
Soukas A, Socci N D, Saatkamp B D, Novelli S, Friedman J M. (2001). Distinct transcriptional profiles of adipogenesis in vivo and in vitro. J Biol. Chem. 276(36), 34167-34174.
Tontonoz P, Hu E, Spiegelman B M. (1994). Stimulation of adipogenesis in fibroblasts by PPAR gamma 2, a lipid-activated transcription factor. Cell 79(7), 1147-1156.
Wasserman F. the development of adipose tissue. In Handbook of Physiology vol. 5 (eds. Renold, a & Cahill, G) 87-100 (American Physiological Society, Washington, D.C., 1965.
Wu Z, Rosen E D, Brun R, Hauser S, Adelmant G, Troy A E, McKeon C, Darlington G J, and Spiegelman B M. (1999). Cross-regulation of C/EBP alpha and PPAR gamma controls the transcriptional pathway of adipogenesis and insulin sensitivity. Mol. Cell. 3(2), 151-158.
Yoo E J, Chung J J, Choe S S, Kim K H, Kim J B. (2006) Down-regulation of histone deacetylases stimulates adipocyte differentiation. J Biol. Chem. 281(10): 6608-6615.
Zhang J, Fu M, Cui T, Xiong C, Xu K, Zhong W, Xiao Y, Floyd D, Liang J, Li E, Song Q, and Chen Y E. (2004). Selective disruption of PPAR gamma 2 impairs the development of adipose tissue and insulin sensitivity. Proc Natl Acad Sci USA. 101(29):10703-10708.

1. A method of treating a disorder associated with increased or decreased fat storage in a mammal comprising modulating the activity or level of the SLUG protein or the SLUG gene in the mammal.

2. The method according to claim 1, wherein the activity of the SLUG protein is increased or the transcription or translation of the SLUG gene is increased.

3. The method according to claim 1, wherein the activity of the SLUG protein is decreased or the transcription or translation of the SLUG gene is decreased.

4. The method according to any preceding claim comprising administering the SLUG protein, a modified form of the SLUG protein or a functional equivalent of the SLUG protein to the mammal.

5. The method according to claim 4, wherein the functional equivalent of the SLUG protein shows an increase in one or more of the activities possessed by the normal SLUG protein.

6. The method according to claim 4, wherein the functional equivalent of the SLUG protein shows a decrease in one or more of the activities possessed by the normal SLUG protein.

7. The method according to claim 1, comprising administering a compound that modulates the activity of the SLUG protein, or modulates transcription and/or translation of the SLUG gene to the mammal.

8. The method according to claim 7, wherein the compound upregulates the activity of the SLUG protein or upregulates the transcription and/or translation of the SLUG gene.

9. The method according to claim 7, wherein the compound downregulates the activity of the SLUG protein or downregulates the transcription and/or translation of the SLUG gene.

10. The method according to claim 1, wherein the disorder is obesity.

11. The method according to claim 1 wherein the disorder is anorexia.

12. The method according to claim 1, wherein the disorder is lipodystrophy.

13. The method according to claim 10, wherein the compound is selected from the group consisting of antisense SLUG mRNA, ribozymes, triple helix molecules, small interference RNA (siRNA), antibodies anti-SLUG, enzymes or proteins which regulate the activity of SLUG protein, and mixtures thereof.

14. The method according to claim 11, wherein the compound is selected from BCR-ABL protein, c-Kit protein, FGF1 protein, VEGF165, SCF, ngn3 protein, FKHR protein, PAX3 and beta catenin.

15. Use of the SLUG protein, a modified form of the SLUG protein or a functional equivalent of the SLUG protein for treating or preventing a disorder associated with increased or decreased fat storage in a mammal.

16. The use according to claim 15, wherein the functional equivalent of the SLUG protein shows an increase in one or more of the activities possessed by the normal SLUG protein.

17. The use according to claim 15, wherein the functional equivalent of the SLUG protein shows a decrease in one or more of the activities possessed by the normal SLUG protein.

18. Use of a compound that modulates the activity of the SLUG protein, or modulates transcription and/or translation of the SLUG gene for treating or preventing a disorder associated with increased or decreased fat storage in a mammal.

19. The use according to claim 18, wherein the compound upregulates the activity of the SLUG protein or upregulates the transcription and/or translation of the SLUG gene.

20. The use according to claim 18, wherein the compound downregulates the activity of the SLUG protein or downregulates the transcription and/or translation of the SLUG gene

21. The use of a protein according to claim 15 or a compound according to claims 18, wherein the disorder is anorexia.

22. The use of a protein according to claim 15 or a compound according claim 18, wherein the disorder is obesity.

23. The use of a protein according to claim 15, wherein the disorder is lipodystrophy.

24. The use according to claim 22, wherein the compound is selected from the group consisting of antisense SLUG mRNA, ribozymes, triple helix molecules, small interference RNA (siRNA), antibodies anti-SLUG, enzymes or proteins which regulate the activity of SLUG protein, and mixtures thereof.

25. The use according to claim 21, wherein the compound is selected from BCR-ABL protein, c-Kit protein, FGF1 protein, VEGF165, SCF, ngn3 protein, FKHR protein, PAX3 and beta catenin.

26. A method for screening for a compound that modulates the activity of the SLUG protein or a functional equivalent of the SLUG protein or modulates the level of transcription or translation from the SLUG gene, comprising administering a candidate compound to a test non-human mammal and monitoring the effect on fat storage in the test non-human mammal.

27. The method according to claim 26, wherein monitoring the effect on fat storage in the test non-human mammal comprises assessing the amount of adipose tissue in the test non-human mammal and optionally:
(i) comparing the amount of adipose tissue in the first test non-human animal to a second test non-human mammal of the same species; or
(ii) comparing the amount of adipose tissue in the first test non-human animal to a second test non-human mammal of the same species which has been administered a placebo; or
(iii) comparing the amount of adipose tissue in the first test non-human mammal before and after administration of the candidate compound.

28. The method of claim 27, wherein the adipose tissue is white adipose tissue.

29. The method of claim 26 wherein the first and/or second test non-human mammal is a transgenic or knockout non-human mammal that has been transformed to express higher, lower or absent levels of a SLUG polypeptide.

30. The method of claim 29, wherein the transgenic or knockout non-human mammal comprises in its genome a transgene that comprises a nucleic acid sequence encoding the SLUG protein, wherein the expression of said transgene can be regulated exogenously by an effector substance.

31. The method of claim 30, wherein expression of the transgene is tetracycline-regulated.

32. The method of claim 29, wherein said transgenic or knockout non-human mammal suffers a disorder associated with increased or decreased fat storage.

33. The method according to claim 32, wherein the disorder is obesity, anorexia or a lipodystrophy.

34. The method of claim 26, wherein the first and/or second test non-human mammal is a rodent.

35. The method of claim 34, wherein the rodent is a mouse or a rat.

36. A method for screening for a compound that modulates the level of transcription or translation from the SLUG gene, or modulates the activity of the SLUG protein or a derivative of the SLUG protein, comprising contacting a cell with a candidate compound and monitoring the amount of lipid accumulation in the cell.

37. The method of claim 36, wherein the cell is derived from a transgenic or knockout non-human mammal as characterised in claim 29.

38. The method of claim 37, wherein the cell is an embryonic fibroblast cell.

39. The method of claim 38, wherein the cell is a mouse embryonic fibroblast. (MEF) cell.

40. The method of claim 36, wherein the cell is a human embryonic fibroblast (HEF) cell.

41. The method according to claim 36, wherein monitoring the effect on the cell optionally comprises monitoring the level of PPARγ2 in the cell.

42. A compound that modulates the level of transcription or translation from the SLUG gene, or modulates the activity of the SLUG protein or a derivative of the SLUG protein obtained or obtainable by the method of claim 26.

43. A pharmaceutical composition comprising a protein as defined in claim 4.

44. A compound according to claim 42 for use as a medicament.

45. Use of a compound according to claim 42 in the manufacture of a medicament for treating or preventing a disorder associated with increased or decreased fat storage.

46. The use according to claim 45, wherein the disorder is obesity, anorexia or lipodystrophy.

47. A method for altering fat storage in a mammal comprising administering a compound according to claim 42.
